(19) United States
(12) Patent Application Publication
(10) Pub. No.: US 2015/0307940 A1
(43) Pub. Date: Oct. 29, 2015

(54) MOLECULAR SIGNATURE OF CUTANEOUS PIGMENTARY SPOTS, ASSOCIATED WITH THE EXTRACELLULAR MATRIX

(71) Applicants: Francoise BERNERD, Nice (FR); Christine DUVAL, Deuil-La-Barre (FR); Olivier DE LACHARRIERE, Paris (FR); Stéphanie NOUVEAU, Boulogne (FR); Xavier MARAT, Paris (FR)

(72) Inventors: Francoise BERNERD, Nice (FR); Christine DUVAL, Deuil-La-Barre (FR); Olivier DE LACHARRIERE, Paris (FR); Stéphanie NOUVEAU, Boulogne (FR); Xavier MARAT, Paris (FR)

(73) Assignee: L'OREAL, Paris (FR)

(21) Appl. No.: 14/111,802

(22) PCT Filed: Apr. 20, 2012
(86) PCT No.: PCT/FR2012/050860

§ 371 Date: Jan. 22, 2014

Related U.S. Application Data
(60) Provisional application No. 61/494,441, filed on Jun. 8, 2011.
(60) Provisional application No. 61/494,438, filed on Jun. 8, 2011.

(30) Foreign Application Priority Data
Apr. 22, 2011 (FR) 1153533
Apr. 22, 2011 (FR) 1153537

(51) Int. Cl. C12Q 1/68; A61K 31/4439; A61K 31/4375; A61N 7/00; A61K 36/00; A61K 31/4745; A61K 31/343; A61K 35/32; A61K 31/517

(52) U.S. Cl. CPC C12Q 1/6883; A61K 35/32; A61K 31/4439; A61K 31/4375; A61K 31/517; A61K 36/00; A61K 31/4745; A61K 31/343; A61N 7/00; C12Q 2600/16; C12Q 2600/158; C12Q 2600/112; C12Q 2600/106; A61N 2007/0034

(57) ABSTRACT

The present invention concerns a molecular signature of cutaneous pigmentary spots, comprising the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1, and various applications of this signature. In particular, the invention concerns a method for characterizing a known or suspected pigmentary spot in a human being, comprising comparing the levels of expression in skin samples obtained from said spot and from adjacent undamaged skin, of at least one dermal gene linked to the extracellular matrix selected from the list constituted by the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1. The invention also concerns methods for evaluating the efficacy of a pigmentary spot treatment, cosmetic and therapeutic methods for the treatment of pigmentary spots, and various modulators for said genes, and their use.

The present invention pertains to the cosmetics field and relates to the skin. More generally, it falls within the purview of the characterization of pigmentary spots on the skin and to the treatment of such spots.

Skin colour is principally due to the presence of a pigment, melanin, in the epidermis. Melanin is synthesized by specific dendritic cells located in the basal layer of the epidermis, namely the melanocytes. Melanogenesis takes place in organelles, the melanosomes which, when loaded with melanin, are transferred to neighbouring epidermal cells, the keratinocytes, via the dendrites. Skin colour, or constitutive pigmentation, varies from individual to individual as a function of the quantity of melanin produced as well as the chemical nature of melanins. Melanins are macromolecules formed from tyrosine (eumelanin) or from tyrosine and cysteine (pheomelanin). The synthesis mechanisms employ enzymes the principal ones of which are tyrosinase and tyrosinase-related protein (Tyrp-1). The pigmentation of the skin is naturally stimulated by exposure to the sun, i.e. the phenomenon of tanning.

However, there are situations where the process of pigmentation is altered, which can result in pigmentation defects, hypopigmentations (vitiligo, albinism) or, in contrast, to an excess of pigmentation, hyperpigmentations. Benign hyperpigmentary disorders, which are characterized by an abnormal accumulation (apart from tanning) of melanin, which may be cited include actinic lentigo, melasma, acne-related pigmentation, post-inflammatory pigmentation, lime disease, pigmentation linked to poison ivy or benign facial dyschromia.

Actinic lentigo, also known as senile or solar lentigo, liver spots, old age spots, or “senile freckles”, is by far the most frequent of pigmentary lesions. This type of lesion appears on zones of the skin which have been photoexposed, such as the face, the back of the hands, the upper limbs and in particular the dorsal face of the forearms, the back and in particular the top of the back. They generally affect individuals from the age of 40.

Macroscopically, actinic lentigines are represented by benign dark to light brown coloured pigmented maculae; they have distinct but irregular edges. They vary greatly in size and may be from a few millimetres to more than two centimetres.

Despite its high frequency, only a few studies have been published on the physiology and pathogenesis of actinic lentigines. Based on the rare existing histological studies, there is known to be an increase in the overall epidermal melanin load, more particularly in the basal layer. There is also an elongation of epidermal ridges which may take on a club-shaped appearance in the papillary dermis [Montagna 1980, ber Rahman 1996, Andersen 1997, Cario-Andre 2004]. Finally, anastomoses may occur between adjacent ridges, producing a bridged or net-like appearance.

Pigmentary incontinence with the presence of melanin and melanophages may also exist in the dermis.

### Current Treatments:

Benign cutaneous pigmentary disorders are generally considered to be unattractive.

Because of the proven association between the appearance of actinic lentigines and chronic exposure to the sun, preventing them from appearing generally involves the topical application of photoprotective substances such as sunscreens. In the case of “curative” treatments, many procedures, principally cosmetic in intent, have thus been developed in order to attempt to eliminate them or reduce their presence. Eliminating or reducing the presence of these problems is usually based on the application of depigmenting treatments based on reducing melanin synthesis activity in the melanocytes. Depigmenting molecules interfere with one or more steps of melanogenesis. One of the principal pathways used today is based on the inhibition of tyrosinase, one of the key enzymes in the melanogenesis process. The aim of those treatments is to reduce or even to stop the synthesis of pigment.

The principal known depigmenting substances are hydroquinone and its derivatives, kojic acid, arbutin, iminophenols, ascorbic acid and its derivatives, a combination of carnitine and quinone, aminophenol derivatives, benzothiazole derivatives, natural extracts, corticoids, etc. Exfoliants are also often associated with those active ingredients in order to increase desquamation, and thus to eliminate the melanin present in the stratum corneum more easily.

Another non-cosmetic treatment method consists of destroying the lesions by physical or chemical means using lasers or peeling. However, these are relatively hard-hitting procedures which do not challenge the etiology of the disorder. In the majority of cases, the actinic lentigines reappear a short time after the treatment.

Further, existing treatments suffer from major disadvantages. Depigmenting substances usually suffer from a certain amount of instability, low efficacy at low concentrations, a biological activity which affects other functions, toxic properties or allergizing properties.

Furthermore, because they do not target the basic underlying cause of pigmentation deregulation, the response to the various depigmenting treatments varies widely. Thus, for a given treatment, administered, for example, in a topical manner, a given benign skin pigmentation problem such as an actinic lentigo or melasma might be attenuated or might disappear, while another does not change at all. This variability can be observed between lesions in different individuals, but also in the same individual for different lesions. Thus, benign skin pigmentation problems exist for which no current topical treatment is effective.

Thus, there is a need to develop more effective and less harmful treatments for this type of lesion.

To this end, it is necessary to identify the molecular deregulations and functional dysfunctions that might exist in this type of pigmentary disorder in order to be able to identify novel targets and to select active ingredients that can correct these defects.

The prior art concerning the treatment of pigmentary spots, more particularly actinic lentigines, describes the stimulation, at the genomic or protein level, of molecules which are closely associated with melanocytes and with melanogenesis (melanogenesis enzymes, melanosome protein, key paracrine factors in melanogenesis). It is found in the epidermis or the dermis for the following proteins: tyrosinase, TRP1, DCT, Pmel-17, POMC, ET1, ETBR, SCF, c-KIT, KGF, KGFR, hepatocyte growth factor (HGF), MIA, TRPM1, melan-A, pink eye dilution, P53 and IL1α.

Furthermore, other studies describe molecular or cellular modifications in solar or senile lentigo by analyses of the expression of genes or proteins. However, those various studies do not discuss any mechanisms or general functional dysfunctions underlying the appearance of pigmentary spots.

No documents in the prior art have reliably indicated the molecular deregulations or functional dysfunctions which might exist in this type of pigmentary disorder beyond the molecules closely associated with melanocytes and melanogenesis.

The present inventors have for the first time demonstrated the involvement of dermal genes linked to the extracellular matrix and to the dermoepidermal junction in pigmentary deregulations resulting in pigmentary spots on the skin, and in particular the involvement of genes linked to the TGF-beta-SMAD signalling pathway.

The extracellular matrix carries out a structural role in the skin because of its capacity to provide support and cohesion for tissues and cells and because of its mechanical properties, which mean that it can resist tension (due in particular to the presence of collagens), and compression (in particular due to the presence of proteoglycans).

It plays an important biological role as it is also a major protagonist in the regulation of epidermal and dermal homeostasis. Because, inter alia, of its growth factor and cytokine reservoir properties, it is involved in morphogenesis, proliferation, cell differentiation and tissue repair.

The dermoepidermal junction (DEJ) itself is produced by the basal membrane which is constituted by a sheet of extracellular matrix and which separates the epidermis from the dermis. It constitutes a permeability barrier and regulates molecule exchange, in particular that of nutrients, between the two tissues. It carries out a function of attachment and anchoring of the epidermis to the subjacent matrix and of structural cohesion of the epithelium. It also plays an important role in regulating differentiation and in the migration of epidermal cells as well as the steps of morphogenesis of the epidermis.

Transforming growth factor beta (TGF-β) controls the proliferation, cellular differentiation and other functions in most cells. It plays a role in immunity and cancer.

Certain cells secret TGF-βs and are also receptors for TGF-β. SMADs make up the signalling pathway for TGF-β. They allow signal transduction when TGF-β binds to membrane receptors. The cell responses induced by this pathway may vary for a given cell type depending on the cellular context. It is a highly dynamic signalling system.

In the canonical pathway, TGF-β binds to a type II membrane receptor which forms a bidimeric complex with a second type I receptor. A type III receptor (betaglycan) aids this process of binding by capturing TGF-β and presenting it to the bidimeric receptor complex. The type II receptor, bound to TGF-β, phosphorylates the type I receptor which propagates the signal by in turn phosphorylating cytosolic proteins, namely the SMAD family (SMAD 2 and 3). The SMAD family regulated via the type I receptor (R-SMAD) then binds to a common SMAD, SMAD 4, to make up an activated complex. This complex then enters the cell nucleus where it acts as a transcription factor for multiple genes and results in an activation or repression response.

Specific type I receptors activated by other ligands such as various BMPs (bone morphogenic proteins) activate the signalling pathway via other SMADs (1, 5, 8).

In general, activation of the pathway also results in stimulation of inhibiting SMADs such as SMAD 6 and 7 which provide a negative retro-control.

Among the target genes of the TGF-β pathway are numerous genes coding for molecules of the extracellular matrix, including collagens, elastin, fibronectin, thrombospondins which principally provide a structural role, and simultaneously for matrix remodelling proteins which participate in renewal and degradation of molecules of the matrix.

This pathway also has a stimulating or inhibiting action in the production of numerous growth factors and cytokines by dermal cells.

For the first time, it has been demonstrated by the present inventors that certain genes linked to the extracellular matrix or to the dermoepidermal junction, and in particular linked to the TGF-β-SMAD signalling pathway have levels of transcription which differ significantly between healthy skin and skin obtained from a pigmentary spot, thereby demonstrating the link between deregulation within the extracellular matrix and the dermoepidermal junction, in particular deregulation of TGF-β-SMAD signalling, and modulation of pigmentation, in particular inducing the appearance of pigmentary spots.

It may be considered that the increase or reduction of certain proteins involved in these various pathways, either dependent on TGF-β or on BMPs, might result in increased and/or aberrant signalling which could perturb dermal, junctional homeostasis and have a knock-on effect on the epidermis.

Without wishing to be bound by a particular theory, the modifications in the expression of various genes linked to this extracellular matrix and to the dermoepidermal junction have led the inventors to believe that, because of the role played by the proteins of the matrix and of the dermoepidermal junction, deregulation of these genes, a sign of a modification of the whole dermal compartment, perturbs the homeostasis of the epidermis and generates substantial modifications, in particular in the histological architecture of the epidermis in pigmentary spots (elongation of epidermal ridges in the dermis, for example). As a consequence, the epidermal melanin load is increased, giving rise to pigmentary spots.

The present invention pertains to a molecular signature representative of differences in gene expression existing between skin obtained from a pigmentary spot and adjacent healthy skin, and to different applications and methods exploiting the knowledge of this signature, in particular in order to modulate the pigmentation of the skin in the cosmetic treatment of pigmentary spots or to even out the complexion or to homogenize the colour of the skin. This signature is constituted by the following genes: TGFBR2 (transforming growth factor beta receptor 2 [70/80 kDa]), TGFBI (transforming growth factor beta induced, 68 kDa), BMP2 (bone morphogenic protein 2), SMAD3 (SMAD family member 3), THBS2 (thrombospondin 2), TGFBR3 (transforming growth factor beta receptor III), SEMA5A (sema domain, 7 thrombospondin repeats, semaphorin 5A), SMAD7 (SMAD family member 7), SOSTDC1 (sclerostin domain-containing protein 1), FRAS1 (Fraser syndrome 1), LEPREL1 (leprecan-like 1), MATN2 (matrilin 2), DST (dystonin, also known as BPAG1), PLOD2 (procollagen-lysine, 2-oxoglutarate 5-dioxygenase 2), ITGA2 (integrin alpha 2 (CD49B, sub-unit alpha 2 of VLA-2 receptor)), COL6A3 (collagen type 6, alpha 3), CRTAP (cartilage associated protein, also known as LEPREL3), LAMC1 (laminin gamma 1, formerly LAMB2), LAMB3 (laminin beta 3), LAMA3 (laminin alpha 3), ITGAV (integrin alpha V (vitronectin receptor)), ITGB1 (integrin beta 1) and ACTN1 (actinin alpha 1). The ITGB1 gene may be excluded from this list. The SEMA5A gene and/or the ITGB1 gene may be excluded from this list.

These genes can be grouped functionally as follows:
List A: Genes coding for factors involved in the activation of the TGF-beta-SMAD signalling pathway: TGFBR2, TGFBI, BMP2, SMAD3, THBS2, TGFBR3, SEMA5A, SMAD7 and SOSTDC1, the gene SEMA5A however may optionally be excluded from this list A; and
List B, encompassing the genes FRAS1, LEPREL1, MATN2, DST, PLOD2, ITGA2, COL6A3, CRTAP, LAMC1, LAMB3, LAMA3, ITGAV, ITGB1 and ACTN1. The gene ITGB1 can optionally be excluded from this list B. In list B, the following functional groups can be discerned:
   Genes coding for collagens, components of the extracellular matrix, and more particularly for filamentous collagens of the stroma and for molecules associated with biosynthesis and with collagen assembly: LEPREL1, PLOD2, COL6A3 and CRTAP;
   Genes coding for laminins, adhesive proteins of the extracellular matrix: LAMC1, LAMB3 and LAMA3;
   Genes coding for matrix proteins associated with the basal membrane zone: FRAS1, MATN2 and DST;
   Genes coding for integrins, involved in binding of cells to the extracellular matrix: ITGA2 and ITGAV, optionally with ITGB1; and
   Gene coding for an actin, component of the extracellular matrix: ACTN1.

Preferred genes from the dermal genes TGFBR2, TGFBI, BMP2, SMAD3, THBS2, TGFBR3, SEMA5A, SMAD7 and SOSTDC1, constituting list A, are the genes TGFBR2, TGFBI, BMP2, SMAD3, THBS2 and TGFBR3.

Preferred genes from the dermal genes FRAS1, LEPREL1, MATN2, DST, PLOD2, ITGA2, COL6A3, CRTAP, LAMC1, LAMB3, LAMA3, ITGAV, ITGB1 and ACTN1, constituting list B, are the genes FRAS1, LEPREL1, MATN2, DST, PLOD2 and ITGA2.

“Said genes” means the human genes mentioned here.

Gene lists A and B are also linked by the number of known interactions between the various genes constituting these lists. These links may in particular be demonstrated by a network of interactions as generated, for example, using IPA software (Ingenuity pathway analysis) which, starting from bibliographic data, can be used to show the various direct or indirect links existing between the genes or proteins. An interaction network generated by this IPA software has in fact demonstrated a link, direct or indirect, between the genes and/or proteins TGFBR2, TGFBR3, SMAD3, SMAD7, BMP2, THBS2 (list A) and MATN2, DST, ITGA2, COL6A3, LAMC1, LAMB3, ITGAV, ITGB1, ACTN1 (list B).

Furthermore, various bibliographic references also illustrate direct interactions between the genes mentioned in list A and those mentioned in list B, thereby giving credence to the functional link uniting these two families, in particular between the genes SMAD3 and COL6A3 (Verrecchia et al, 2001), between the genes TGFBR2, THBS2 and COL6A3 (Berking et al, 2001) and between SMAD3 and LAMC1 (Kawata et al, 2002), by way of illustration.

The inventors have in fact demonstrated the significant and reproducible modulation of the level of expression of genes from lists A and B between skin obtained from a pigmentary spot and corresponding healthy skin.

Furthermore, modulation of genes from list A, involved in the TGF-β signalling pathway within hyperpigmentary spots, tends to increase the TGF-β-SMAD signalling. The modulations observed for genes from list A thus demonstrate the link between activation/stimulation of the TGF-β-SMAD signalling pathway and increase of pigmentation, as the inventors verify in the present Example 4.

In a first aspect, the present invention in particular concerns a method for characterizing a cutaneous pigmentary spot. Such a method can be used, inter alia, to confirm the nature of the pigmentary spot in the case in which the latter is already apparent, for example visually to the naked eye. The method can also be used to predict the appearance of a spot when it is not yet observable but only suspected, or to conclude that a person's skin has a tendency to form cutaneous spots or is prone to pigmentation defects, for example when no spots can yet be seen.

The cutaneous pigmentary spots concerned are hyperpigmentary spots or hyperpigmented spots corresponding to an excess of pigment, or hypopigmentary spots or hypopigmented spots corresponding to pigmentation defects. Particular hypopigmentations which can be envisaged in the context of the present invention are vitiligo and albinism. Examples of benign hyperpigmentary disorders which can be envisaged in the context of the invention, characterized by an abnormal accumulation of melanin (apart from tanning), are actinic lentigo, melasma, acne-related pigmentation, post-inflammatory pigmentation, lime disease, pigmentation linked to poison ivy or again benign facial dyschromias. “Pigmentary spots” in the present invention also encompasses faults, imperfections or irregularities of pigmentation rendering the complexion non-uniform or the skin colour non-homogeneous.

The pigmentary spots in question are preferably pigmentary spots on human skin. However, disorders of the extracellular matrix and the TGF-β-SMAD signalling pathway in the dermis demonstrated by the present inventors are entirely general, and so similar methods could be envisaged for other animal species also affected by pigmentary spots. In this case, the various methods, uses or compositions of the invention will be employed with the genes of the species under consideration, in an orthologous manner to the human genes of the invention.

The method comprises comparing levels of expression in skin obtained from said spot and from undamaged skin, preferably adjacent thereto from the same individual, of at least one dermal gene linked to the extracellular matrix selected from the list constituted by the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1. Alternatively, the dermal gene may be selected from list A constituted by genes linked to the TGF-β-SMAD signalling pathway; or from list B constituted by the genes FRAS1, LEPREL1, MATN2, DST, PLOD2, ITGA2, COL6A3, CRTAP, LAMC1, LAMB3, LAMA3, ITGAV and ACTN1. In some embodiments, SEMA5A is excluded from these lists. Alternatively or in addition, this could be the same for the gene ITGB1.

The genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1 are the genes of the invention. They play a role in the structure or renewal of the extracellular matrix; in particular they are genes involved in the activation of the TGF-β-SMAD signalling pathway, or of genes coding for components of the extracellular matrix or of genes coding for proteins involved in the synthesis of components of this matrix, or indeed linked to renewal of this connective matrix, or of genes coding for matrix proteins associated with the dermoepidermal junction and to the basal membrane zone. The link between the genes of the invention and the extracellular matrix pertains to one of the roles mentioned.

The term “genes of the invention”, also means the dermal genes from list A; or indeed also the dermal genes from list B.

The gene SEMA5A and/or the gene ITGB1 may be excluded from these lists.

Furthermore, said genes of the invention are known as dermal genes because they are genes expressed mainly in the dermis and giving rise to characteristic proteins of the dermal compartment or of the dermoepidermal junction as regards its dermal face; this is in contrast, for example, to keratinocytary proteins, to intracellular proteins or to epidermal proteins in particular, such as proteins expressed preferentially in the stratum corneum.

The levels of expression of at least one of the genes of the invention are measured in the skin obtained from the suspected or known spot and from the adjacent undamaged skin. Preferably, the levels are measured on samples of skin removed from the spot and from an adjacent undamaged zone. The samples are skin biopsies, for example. Biopsies a few millimetres in diameter are sufficient, for example a 2 mm or a 3 mm diameter biopsy. Complete excision of a lesion may also be envisaged.

The term “levels of expression” of the genes of the invention means the levels of expression within the cells of the skin dermis or of the sample being studied.

The undamaged zone is preferably an adjacent zone as close as possible to the spot but at a sufficient distance for the zone or sample not to contain any cells which might belong to the pigmentary spot. Preferably, the adjacent undamaged zone is a zone which has been exposed to light and sun in a manner comparable to the pigmentary spot zone. Alternatively, the undamaged zone may come from a symmetrical zone on the other side of the subject in an identical position; as an example, in the case of a spot on the left hand, the undamaged zone may be the corresponding zone on the right hand. In this case, the undamaged zone is not strictly speaking an adjacent zone. The term “undamaged” means a zone which does not have any pigmentary spots, or pigmentary irregularities, preferably a homogeneous zone in terms of pigmentation.

Because the undamaged zone acts as a reference, it must in all cases also be as comparable as possible to the zone of the spot, but free of a pigmentary defect.

The term “level of expression of a gene” as used in the present description preferably means the degree of transcription of said gene. However, its level of expression may also be translated as meaning its degree of translation, assuming however that it is a gene coding for a protein. This is the case for the genes of the invention.

Concerning the evaluation of the degree of transcription of the selected gene, this may be carried out in different manners which are familiar to the skilled person, directly or indeed after reverse transcription. The degree of transcription may in particular be evaluated by using RNA or DNA arrays commercially available for this purpose. One possible evaluation method is described in the experimental section.

It is also important to note that the method of the invention involves comparing the levels of expression of at least one of the genes of the invention, or at least one of the genes from list A or from list B. For this reason, it may be sufficient to quantitatively or qualitatively evaluate the difference between the two levels of expression without ever individually evaluating and quantifying each of the levels of expression.

The levels of expression of at least one of the genes of the invention may be evaluated by reference to or after normalization with the level of expression of other genes the level of expression of which is assumed to be substantially identical in the spot and in the selected undamaged skin zone. Such genes for normalization are well known to the skilled person and may depend on the zone of the body where the spot is located. By way of example, the following genes may be cited as susceptible of being used for normalization of the levels of expression of the genes of the invention, coding for:

Ribosomal protein L13a (RPL13A), beta-2-microglobulin (B2M), ribosomal protein S9 (RPS9), ribosomal protein S28 (RPS28) and glyceraldehyde-3-phosphate dehydrogenase (GAPDH).

The method of the invention is preferably carried out in vitro, or indeed ex vivo.

In one embodiment of the method of the invention, the pigmentary spot is a non-pathological spot, which is benign, in particular in contrast to pathological lesions such as nevi; it may be an irregularity in the pigmentation of the skin.

Preferably, a method in accordance with the invention comprises comparing the levels of expression of at least two distinct genes taken from the genes of the invention, preferably of at least three distinct genes, or even five distinct genes. It is also possible to compare the levels of expression of at least 6 genes, or even of at least 8 distinct genes or even 10, 12, 15, 20 or even all of the genes of the invention.

In one embodiment, the distinct genes are selected from the genes TGFBR2, TGFBI, BMP2, SMAD3, THBS2, TGFBR3, SEMA5A, SMAD7 and SOSTDC1, or indeed from TGFBR2, TGFBI, BMP2, SMAD3, THBS2, TGFBR3, SMAD7 and SOSTDC1.

When two genes are selected, they are preferably selected from the following list of 6 preferred genes: TGFBR2, TGFBI, BMP2, SMAD3, THBS2 and TGFBR3. As an example, the selected genes may be TGFBR2 and TGFBI, or TGFBR2 and BMP2, or TGFBI and BMP2, or TGFBR2 and TGFBR3. Any paired combinations from the 6 preferred genes are preferred combinations for carrying out the invention. Similarly, any combination involving one of the 6 preferred genes and one of the other genes from list A are particularly preferred.

The following combinations are envisaged for combinations of 3 genes: TGFBR2, TGFBI and BMP2; TGFBR2, TGFBI and SMAD3; TGFBR2, TGFBI and TGFBR3; SMAD3, SEMA5A and SMAD7; TGFBR2, BMP2 and SOSTDC1; and TGFBI, BMP2 and SMAD3.

The following combinations are envisaged for combinations of 5 genes of the present invention: TGFBR2, TGFBI, BMP2, SMAD3 and THBS2; TGFBR2, TGFBI, BMP2, SMAD3 and TGFBR3; and TGFBR2, TGFBI, BMP2, THBS2 and TGFBR3.

A particular combination is that comprising the genes TGFBR2, TGFBI, BMP2, SMAD3, THBS2, TGFBR3, SEMA5A and SMAD7, which have in common the fact that they are modulated in the same manner; in particular, they are overexpressed in hyperpigmentary spots and the fact that they are involved in the TGF-beta-SMAD signalling pathway. Other combinations can also be envisaged in the context of the present invention, in particular combinations comprising at least one gene selected from TGFBR2, TGFBI, THBS2 and TGFBR3; and at least one gene selected from SMAD3, SEMA5A and SMAD7.

The genes TGFBR2, TGFBI, BMP2, SMAD3, THBS2, TGFBR3, SEMA5A, SMAD7 and SOSTDC1 have in common the fact that they belong to the TGF-β-SMAD signalling pathway and are modulated in a cooperative manner in pigmentary spots, i.e. the genes TGFBR2, TGFBI, BMP2, SMAD3, THBS2, TGFBR3, SEMA5A and SMAD7, which are positively involved in this signalling pathway are all modulated in the same sense and they are modulated in the reverse sense to the gene SOSTDC1, which is a BMP antagonist and thus negatively involved in this signalling pathway.

In another embodiment, the distinct genes are selected from genes from list B or from the following 6 preferred genes: FRAS1, LEPREL1, MATN2, DST, PLOD2 and ITGA2. As an example, the selected genes may be FRAS1 and LEPREL1, or indeed FRAS1 and MATN2, or indeed LEPREL and MATN2, or indeed DST and PLOD2. Any paired combinations of the 6 preferred genes FRAS1, LEPREL1, MATN2, DST, PLOD2 and ITGA2, are preferential combinations for carrying out the invention. Similarly, any combinations involving one of the 6 preferred genes and one of the other genes from list B are particularly preferred.

The following combinations are envisaged for combinations of 3 genes: FRAS1, LEPREL1 and MATN2; FRAS1, LEPREL1 and DST; FRAS1, LEPREL1 and PLOD2; FRAS1, MATN2, and PLOD2; LEPREL1, MATN2 and PLOD2.

The following combinations are envisaged in the present invention for combinations of 5 genes: FRAS1, LEPREL1, MATN2, DST and PLOD2; FRAS1, LEPREL1, MATN2, DST and ITGA2; FRAS1, LEPREL1, MATN2, PLOD2 and ITGA2.

A particular combination is that constituted by the genes FRAS1, LEPREL1, MATN2, DST, ITGA2, COL6A3, CRTAP, LAMC1, LAMB3, LAMA3, ITGAV and ACTN1, which have in common the fact that they are modulated in the same manner, in particular of being overexpressed in hyperpigmentary spots.

Other combinations can also be envisaged in the context of the present invention, in particular combinations comprising at least one gene selected from those belonging to the collagen family, i.e. from LEPREL1, PLOD2, COL6A3 and CRTAP; at least one gene selected from those belonging to the laminin family, i.e. from LAMC1, LAMB3 and LAMA3; at least one gene selected from those coding for matrix proteins associated with the basal membrane zone, i.e. from FRAS1, MATN2 and DST; at least one gene from the integrin family, i.e. from ITGA2 and ITGAV, and optionally ITGB1; and the gene ACTN1.

Alternatively, in a preferred embodiment of the invention, the gene the level of expression of which is compared between damaged skin and healthy skin, preferably adjacent, is selected from the sub-group constituted by the genes LEPREL1, PLOD2, COL6A3 and CRTAP. In fact, these genes have in common the fact that they belong to the collagen family, in particular to the stromal collagen fibril family.

Other combinations can be envisaged in the context of the present invention, in particular combinations comprising at least one gene selected from FRAS1, LEPREL1, MATN2, DST, ITGA2, COL6A3, CRTAP, LAMC1, LAMB3, LAMA3, ITGAV and ACTN1; and the gene PLOD2.

In accordance with yet another embodiment of the invention, other combinations are envisaged, in particular combinations comprising at least one gene selected from those involved in the TGF-beta-SMAD signalling pathway, i.e. from list A, and at least one gene selected from list B, and preferably from FRAS1, LEPREL1, MATN2, DST, PLOD2 and ITGA2. As an example, one envisaged combination is that constituted by at least two genes from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV and ACTN1 which have in common the fact of being overexpressed in hyperpigmentary spots.

Other particular combinations are combinations of 2 genes, one coming from list A, and the other coming from list B, combinations of 3 genes, two from list A and one from list B, or vice versa; combinations of 4 genes with 2 from each of the lists, or indeed three from list A and one from list B, or vice versa.

A particularly preferred combination of 2 genes is the combination of the genes TGFBR2 and MATN2. Particular preferred combinations of 3 genes are the combination TGFBR2, BMP2 and MATN2 and the combination TGFBR2, MATN2 and LEPREL1, Particular preferred combinations of 4 genes are the combination TGFBR2, BMP2, SMAD3 and MATN2; the combination TGFBR2, MATN2, LEPREL1 and PLOD2; and the combination TGFBR2, BMP2, MATN2 and LEPREL1.

All combinations or sub-groups of specific preferred genes in respect of this aspect of the invention are also preferred for the other aspects of the invention.

Carrying out the method of the invention leads to the conclusion that the spot which is suspected or observed is a hyperpigmentary spot if the level of expression is:
higher in the skin obtained from the spot, or in the skin sample obtained from the spot, compared with the level in the adjacent undamaged skin, or in the sample of adjacent undamaged skin, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
lower in the skin obtained from the spot, or in the skin sample obtained from the spot, compared with the level in the adjacent undamaged skin, or in the sample of adjacent undamaged skin, if the gene is selected from SOSTDC1 and PLOD2.

The present inventors have indeed demonstrated the over-expression of the genes TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1 in skin obtained from a hyperpigmentary spot, in particular actinic lentigo, compared with the level of expression in the adjacent undamaged skin. They have also demonstrated the underexpression of the genes SOSTDC1 and PLOD2 in skin obtained from a hyperpigmentary spot, in particular actinic lentigo, compared with the level of expression in the adjacent undamaged skin.

In contrast, carrying out the method of the invention leads to the conclusion that the suspected or observed spot is a hypopigmentary spot if the level of expression is:
lower in the skin obtained from the spot, or in the skin sample obtained from the spot, compared with the level in the adjacent undamaged skin, or in the sample of adjacent undamaged skin, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
higher in the skin obtained from the spot, or in the skin sample obtained from the spot, compared with the level in the adjacent undamaged skin, or in the sample of adjacent undamaged skin, if the gene is selected from SOSTDC1 and PLOD2.

The term “higher” or “lower” means a difference in the levels of expression which is statistically significant, higher than background noise and reproducible. As an example, the difference in the level of expression is at least 10%, i.e. if the level of expression of a gene of the invention in undamaged skin is fixed at 1, the degree of modulation is at least 1.1 for a gene which is overexpressed in the lesional skin and at most 0.9 for a gene which is underexpressed in the lesional skin.

Preferably, the method of the invention is carried out with at least two genes, one belonging to the category of genes overexpressed in the hyperpigmentary spots, and underexpressed in hypopigmentary spots, and the other belonging to the category of genes modulated in the reverse manner to the first in the pigmentary spots. Preferably, the method is carried out with at least three genes: two genes selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and one gene selected from SOSTDC1 and PLOD2, modulated in the reverse manner to the first two in the pigmentary spots, or indeed two genes selected from TGFBR2, TGFBI, BMP2, SMAD3, THBS2, TGFBR3, SEMA5A and SMAD7, and the gene SOSTDC1; or indeed two genes selected from FRAS1, LEPREL1, MATN2, DST, ITGA2, COL6A3, CRTAP, LAMC1, LAMB3, LAMA3, ITGAV, ITGB1 and ACTN1 and the gene PLOD2, modulated in the reverse manner to the first two in pigmentary spots.

Preferably, in the context of this characterization method, the level of expression of one of the genes of the invention is compared in the skin of a Caucasian type individual. Preferably, said individual is at least forty years of age, preferably at least fifty years of age or even sixty years of age. The individual under consideration in the context of the present invention is preferably female. As detailed above, the level of expression of one or more genes of the invention may be compared within a skin sample from said individual.

Further, the present inventors have also demonstrated the differential modulation of other dermal genes between skin obtained from a pigmentary spot and undamaged skin, preferably adjacent. The inventors have in particular demonstrated the modulation of the following dermal genes, linked to the extracellular matrix:
genes EFEMP1, ECM1, ASPN, HS3ST6, PAPLN, CHSY1 and FLRT2 coding for proteins of the family of extracellular proteoglycans and glycoproteins;
genes MXRA5, LYZ, CTSL2, PLAU and TIMP1 coding for proteins linked to matrix remodelling.

As a consequence, the characterization method in accordance with the present invention also comprises comparing levels of expression in skin obtained from said spot and in undamaged skin, preferably adjacent, of at least one dermal gene selected from the list constituted by the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1 and of at least one second gene selected from the following list of genes: EFEMP1, ECM1, ASPN, HS3ST6, PAPLN, CHSY1, FLRT2, MXRA5, LYZ, CTSL2, PLAU and TIMP1; for example, the second gene is selected from the following list of genes EFEMP1, ECM1, ASPN, HS3ST6, PAPLN, CHSY1, FLRT2 or indeed from the following list of genes: MXRA5, LYZ, CTSL2, PLAU and TIMP1.

Preferred genes from the list of dermal genes EFEMP1, ECM1, ASPN, HS3ST6, PAPLN, CHSY1, FLRT2 are the genes EFEMP1, ECM1, ASPN, HS3ST6, PAPLN and CHSY1; MXRA5, LYZ, CTSL2, PLAU and TIMP1 are also preferred genes.

Preferably, the second gene is selected from the genes EFEMP1, ECM1, ASPN, HS3ST6, PAPLN, CHSY1 and FLRT2, and preferably from the genes EFEMP1, ECM1, ASPN, HS3ST6, PAPLN and CHSY1. Alternatively, the second gene is selected from the genes MXRA5, LYZ, CTSL2, PLAU and TIMP1.

In another embodiment, the first gene is selected from list A of dermal genes involved in the TGF-β-SMAD signalling pathway; the second gene is selected as set out above. In accordance with yet another embodiment, the first gene is selected from list B; the second gene being selected as described above.

In one particular embodiment, the method comprises comparing the levels of expression of at least 3 distinct genes, one selected from the genes TGFBR2, TGFBI, BMP2, SMAD3, THBS2, TGFBR3, SEMA5A, SMAD7 and SOSTDC1, a second selected from the genes EFEMP1, ECM1, ASPN, HS3ST6, PAPLN, CHSY1 and FLRT2 and a third selected from the genes MXRA5, LYZ, CTSL2, PLAU and TIMP1. Alternatively, when 3 distinct genes are selected, the first may be selected from genes from list B, the second and the third being selected as described above. In accordance with another alternative, the first gene is selected from the list of genes comprising TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1; optionally, however, SEMA5A is excluded.

In accordance with yet another embodiment, the method comprises comparing the levels of expression of at least 4 distinct genes, the first three being selected as described above and the 4^{th} from the list constituted by FRAS1, LEPREL1, MATN2, DST, PLOD2, ITGA2, COL6A3, CRTAP, LAMC1, LAMB3, LAMA3, ITGAV, ITGB1 and ACTN1.

All combinations of the genes described above are also preferred combinations in the context of the other aspects of the invention.

If one or more supplemental genes are selected from the genes EFEMP1, ECM1, ASPN, HS3ST6, PAPLN, CHSY1, FLRT2, MXRA5, LYZ, CTSL2, PLAU and TIMP1, then the characterization method can provide the result of confirming the presence of a hyperpigmentary spot if the level of expression is:
higher in the skin obtained from the spot, or in the skin sample obtained from the spot, compared with the level in the adjacent undamaged skin, or in the sample of adjacent undamaged skin, if the gene is selected from EFEMP1, ASPN, PAPLN, CHSY1, MXRA5, LYZ, PLAU and TIMP1, and
lower in the skin obtained from the spot, or in the skin sample obtained from the spot, compared with the level in the adjacent undamaged skin, or in the sample of adjacent undamaged skin, if the gene is selected from HS3ST6, FLRT2, ECM1 and CTSL2.

In contrast, the method of the invention can confirm the presence of a hypopigmentary spot if the level of expression is:
lower in the skin obtained from the spot, or in the skin sample obtained from the spot, compared with the level in the adjacent undamaged skin, or in the sample of adjacent undamaged skin, if the gene is selected from EFEMP1, ASPN, PAPLN, CHSY1, MXRA5, LYZ, PLAU and TIMP1, and
higher in the skin obtained from the spot, or in the skin sample obtained from the spot, compared with the level in the adjacent undamaged skin, or in the sample of adjacent undamaged skin, if the gene is selected from HS3ST6, FLRT2, ECM1 and CTSL2.

The method described is also a test method for predicting the formation of cutaneous spots in a subject. In this implementation, the level of expression of at least one, preferably several of the genes of the invention is compared, in a skin sample, to its level of expression in normal skin. A significant modification to the level of expression compared with normal skin means that the skin of the test subject has a tendency to form cutaneous spots.

The term “normal skin” can mean either the skin of a given subject in a zone of the body which is known to be free of spots, for example zones not exposed to the sun, or zones that have a low tendency to form pigmentary spots. It may also mean the mean level of expression of said genes in the skin of persons free of spots and preferably having the same type of skin as the test subject. The normalization genes described above could be used to normalize the levels of expression of the genes of the invention.

In a second aspect, the present invention also concerns a method for evaluating the efficacy of a treatment of spots or pigmentary irregularities, using the signature demonstrated by the inventors. The evaluated treatment may be a treatment intended to attenuate pigmentary spots or any other pigmentation modulation, in particular to even out the complexion, to homogenize the colour of the skin or to combat dyschromias. In a first implementation, this evaluation method comprises a step for comparing the levels of expression in the skin obtained from a pigmentary spot, before and after treatment, of at least one dermal gene selected from the genes of the invention, namely TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1.

In one embodiment, the gene is selected from TGFBR2, TGFBI, BMP2, SMAD3, THBS2, TGFBR3, SEMA5A, SMAD7 and SOSTDC1, or indeed from TGFBR2, TGFBI, BMP2, SMAD3, THBS2, TGFBR3, SMAD7 and SOSTDC1. In accordance with another embodiment, the gene is selected from FRAS1, LEPREL1, MATN2, DST, PLOD2, ITGA2, COL6A3, CRTAP, LAMC1, LAMB3, LAMA3, ITGAV, ITGB1 and ACTN1. SEMA5A and/or ITGB1 are optionally excluded from the lists.

In this evaluation method, the levels of expression of the selected gene or genes are compared in skin obtained from a pigmentary spot or in a corresponding sample before and after treatment. Thus, there is no comparison with a level of expression in healthy undamaged skin.

As was the case for the characterization method in accordance with the first aspect of the invention, the levels of expression are advantageously normalized with the aid of the levels of expression of genes coding for ribosomal protein L13a (RPL13A), beta-2-microglobulin (B2M), ribosomal protein S9 (RPS9), ribosomal protein S28 (RPS28) and glyceraldehyde-3-phosphate dehydrogenase (GAPDH).

Using the evaluation method described, a given treatment is considered to be effective for the treatment of a hyperpigmentary spot if the level of expression is:
lower after treatment compared with the level of expression before treatment, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
higher after treatment compared with the level of expression before treatment, if the gene is selected from SOSTDC1 and PLOD2.

In contrast, using the evaluation method of the invention, a given treatment is considered to be effective for the treatment of a hypopigmentary spot if the level of expression is:
higher after treatment compared with the level of expression before treatment, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
lower after treatment compared with the level of expression before treatment, if the gene is selected from SOSTDC1 and PLOD2.

A treatment will be considered to be without effect if the levels of expression of the selected gene before and after treatment are substantially identical, or indeed if the observed differences are not significant.

When the levels of expression of more than one gene are compared, the treatment is considered to be effective for the treatment of a spot or a pigmentary irregularity if, for the majority of the tested genes, and preferably for all of the tested genes taken individually, the treatment is considered to be effective. For the other selected genes, the treatment must preferably be without effect, but not have the reverse effect.

In accordance with another embodiment, the method for evaluating the efficacy of a treatment of cutaneous pigmentary spots comprises the characterization of a pigmentary spot or irregularity using the method of the invention, before and after treatment, and comparing the differences in the observed levels of expression between the damaged skin of the pigmentary spot or irregularity and healthy skin.

In accordance with this embodiment for evaluating the treatment efficacy, the treatment is considered to be effective if the difference between the levels of expression of the selected gene or genes in the damaged skin obtained from the spot compared with healthy skin, preferably adjacent, is smaller after treatment compared with what it was before the treatment.

When the levels of expression of more than one gene are compared, the treatment is preferably concluded to be effective when for the majority of the selected genes, preferably for all of the selected genes, taken individually, the conclusion is that the treatment is effective.

Preferably, the comparison of the levels of expression of the selected gene or genes is carried out on samples of skin removed from the pigmentary spot.

The treatment under consideration, evaluated using the method of the invention, is not limited to one particular type of treatment. It may be a treatment using a chemical molecule, an active ingredient, a natural extract, in particular an essential oil, a nucleic acid, in particular an interference RNA, a protein complex or any other molecule or combination of molecules. It may also be a treatment using a physical means or waves, in particular electromagnetic waves. It is preferably a topical treatment, but it might also involve evaluating the efficacy of treatments which are administered orally, by injection or by any other administration means.

The tested treatment may be intended to attenuate a cutaneous spot or cause it to disappear, to modulate skin pigmentation, to even out the complexion, to homogenize the colour of the skin or to attenuate dyschromias.

Particularly preferred treatments in the context of this invention are cosmetic treatments, more particularly topical cosmetic treatments. In this case, the pigmentary spot under consideration is a non-pathological pigmentary spot or irregularity, for example an actinic, solar or senile lentigo.

Using the methods of the invention, it is also possible to evaluate the efficacy of the combination of several treatments. It is in fact possible to evaluate combinations that are best in restoring the levels of expression of one, some or all of the genes of the invention whether from list A or from list B, as they are expressed in undamaged skin.

Using the evaluation methods described above, it is possible to evaluate the efficacy of a novel envisaged treatment, or also to quantify or qualify the efficacy of extant treatments against pigmentary spots, whether they are hyperpigmentary or hypopigmentary. By this means, it is also possible to envisage combinations of treatments that might be particularly effective, synergistic or complementary.

As explained for the characterization methods in accordance with the first aspect of the invention, the levels of expression of more than one gene are preferably compared, preferably of at least two, three, five, six, eight, ten, twelve, fifteen, eighteen or even all of the genes of the invention, or even genes from list A or indeed the genes from list B.

Particularly preferred genes or combinations of genes have already been set out in respect of the first aspect of the invention; the same genes or combinations are preferred in this aspect. In particular, the selected gene is more particularly selected from the genes TGFBR2, TGFBI, BMP2, SMAD3, THBS2, TGFBR3, SEMA5A, SMAD7 and SOSTDC1, and most particularly from the genes TGFBR2, TGFBI, BMP2, SMAD3, THBS2 and TGFBR3, and any two by two or three by three combinations from these lists are particularly preferred. In accordance with another embodiment, the gene is selected from genes from list B, and in particular from the list of genes FRAS1, LEPREL1, MATN2, DST, PLOD2 and ITGA2, and any two by two or three by three combinations from these lists are particularly preferred.

Furthermore, because of the complementary results obtained by the inventors concerning other dermal genes the level of expression of which is modulated in the pigmentary spot, the evaluation methods as described are preferably carried out with:
at least one gene selected from the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1, and even preferably from the genes from list A or from the genes TGFBR2, TGFBI, BMP2, SMAD3, THBS2 and TGFBR3, or from the genes from list B, or again from the genes FRAS1, LEPREL1, MATN2, DST, PLOD2 and ITGA2 and
at least one second gene selected from the genes EFEMP1, ECM1, ASPN, HS3ST6, PAPLN, CHSY1, FLRT2, MXRA5, LYZ, CTSL2, PLAU and TIMP1. Alternatively, the second gene may be selected from the genes EFEMP1, ECM1, ASPN, HS3ST6, PAPLN, CHSY1, FLRT2 or indeed from the genes MXRA5, LYZ, CTSL2, PLAU and TIMP1.

Preferred genes from among the dermal genes EFEMP1, ECM1, ASPN, HS3ST6, PAPLN, CHSY1, FLRT2 are the genes EFEMP1, ECM1, ASPN, HS3ST6, PAPLN and CHSY1.

If one or more supplemental genes are selected from the genes EFEMP1, ECM1, ASPN, HS3ST6, PAPLN, CHSY1, FLRT2, MXRA5, LYZ, CTSL2, PLAU and TIMP1, then the evaluation method will consider a treatment to be effective for the treatment of hyperpigmentary spots if the level of expression is:
lower after treatment, compared with the level of expression before treatment, if the gene is selected from EFEMP1, ASPN, PAPLN, CHSY1, MXRA5, LYZ, PLAU and TIMP1, and
higher after treatment, compared with the level of expression before treatment, if the gene is selected from HS3ST6, FLRT2, ECM1 and CTSL2.

In contrast, the evaluation method will consider a treatment to be effective for the treatment of hypopigmentary spots if the level of expression is:
higher after treatment, compared with the level of expression before treatment, if the gene is selected from EFEMP1, ASPN, PAPLN, CHSY1, MXRA5, LYZ, PLAU and TIMP1, and
lower after treatment, compared with the level of expression before treatment, if the gene is selected from HS3ST6, FLRT2, ECM1 and CTSL2.

The skin sample preferably derives from a Caucasian type human being, preferably at least forty years of age, preferably at least fifty years of age, or even at least sixty years of age.

Preferably, in the context of the present invention and in particular for the evaluation method described, it is a treatment of hyperpigmentary spots, and highly preferably of actinic, solar or senile lentigo.

The method for evaluating the efficacy of a treatment of the present invention is preferably carried out in vitro or ex vivo. It can also be carried out in vivo. The skin sample should desirably be removed from the same pigmentary spot before treatment and after treatment or from a pigmentary spot very close thereto if the size of the spot means that samples cannot easily be obtained before and after treatment.

The present invention also concerns an in vitro method for evaluating the efficacy of a treatment of pigmentary spots; such a method comprises comparing, before and after treatment, the level of expression, in a cellular model representative of the skin, of at least one dermal gene selected from the list constituted by the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1, or the level of expression or activity of an expression product of said selected gene. Alternatively, the gene may be selected from list A of dermal genes linked to the TGF-beta-SMAD signalling pathway, with or without SEMA5A, or indeed from list B, or indeed from the various sub-groups of this latter list, namely from LEPREL1, PLOD2, COL6A3 and CRTAP, or indeed from LAMC1, LAMB3 and LAMA3, or indeed from FRAS1, MATN2 and DST, or again from ITGA2, ITGAV and ITGB1, or between ITGA2 and ITGAV.

The cellular model may be any type considered by the skilled person to be appropriate. In particular, it may be a mono or co-culture cellular model or a three-dimensional model of reconstructed skin, or indeed skin cultivated ex vivo. Cellular models also exist which are representative of pigmentary spots, more particularly of actinic lentigines, which may be used in the context of this method. Such cellular models do not need to mimic the pigmentary spots (or lentigo) completely, but do have to mimic biological events, morphological characteristic or pigmentary characteristics observed in pigmentary spots, in particular lentigo. Such in vitro models are well known to the skilled person.

In accordance with a preferred embodiment of the in vitro method described above, it comprises comparing the levels of expression of at least two genes, preferably at least three, five or six genes of the invention, as explained for the other evaluation methods of the invention, said genes being selected from the genes of the invention, or indeed from the genes from list A, with or without SEMA5A, or indeed from the genes from list B, with or without ITGB1. Most particularly preferred genes are the genes TGFBR2, TGFBI, BMP2, SMAD3, THBS2 and TGFBR3, as well as the genes FRAS1, LEPREL1, MATN2, DST, PLOD2 and ITGA2.

Similarly, as explained for the other evaluation methods, they are preferably carried out with at least two genes:
one being selected from the genes TGFBR2, TGFBI, BMP2, SMAD3, THBS2, TGFBR3, SEMA5A, SMAD7 and SOSTDC1, or indeed from the genes from list B, or again from TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1, and
the second being selected from the genes EFEMP1, ECM1, ASPN, HS3ST6, PAPLN, CHSY1, FLRT2, MXRA5, LYZ, CTSL2, PLAU and TIMP1, preferably from the genes EFEMP1, ECM1, ASPN, HS3ST6, PAPLN, CHSY1 and FLRT2, or indeed from the genes MXRA5, LYZ, CTSL2, PLAU and TIMP1.

Particularly preferably, the first gene is selected from TGFBR2, TGFBI, BMP2, SMAD3, THBS2 and TGFBR3, or indeed from FRAS1, LEPREL1, MATN2, DST, PLOD2 and ITGA2.

Concerning the evaluation methods of the invention, in one preferred embodiment, the first gene is selected from the sub-group constituted by the genes LEPREL1, PLOD2, COL6A3 and CRTAP, belonging to the collagen family, in particular to the stromal collagen fibril family.

In accordance with another embodiment, the first gene is selected from the group constituted by the genes LAMC1, LAMB3 and LAMA3, coding for laminins, and being overexpressed in hyperpigmentary spots.

In accordance with another embodiment, the first gene is selected from the group constituted by the genes FRAS1, MATN2 and DST, coding for matrix proteins associated with the basal membrane zone and being overexpressed in hyperpigmentary spots.

In accordance with yet another embodiment, the first gene is selected from the group constituted by the genes ITGA2, ITGAV and ITGB1, coding for integrins and being overexpressed in hyperpigmentary spots.

Alternatively, the method may be carried with the gene ACTN1, coding for an actin.

All combinations of specific genes described with respect to the other aspects of the invention are also preferred in the context of this aspect.

Further, using these evaluation methods, it is possible to promote a treatment to consumers by highlighting the results obtained with this treatment in the methods for evaluating the efficacy described in the present invention. Thus, the present invention also provides a method that can be used to recommend a product by indicating its effect in a test protocol constituted by a method for evaluating the efficacy as described above. Thus, the invention also concerns a method for promoting a cosmetic product or cosmetic treatment, consisting of highlighting an efficacy, action or property of said product or treatment demonstrated by at least one method operated as described above.

Such a promotion of the product could be carried out using any channel of communication. It can in particular by made by the salesperson, directly at the point of sale, via radio and television, in particular in the context of advertisements. It could also be promoted through the written press, or by means of any other document, in particular for publicity purposes (prospectus). It could also be promoted via the internet or any other suitable data network. It could also be promoted directly on the product, in particular on its packaging or any other explanatory leaflet which could be associated with it. The present invention also concerns a method for screening molecules for the treatment of cutaneous pigmentary spots, comprising carrying out one of the evaluation methods described above in order to determine the efficacy of a treatment based on that molecule.

In a further aspect, the present invention also concerns a cosmetic method for the treatment or prevention of a non-pathological cutaneous pigmentary spot or irregularity of human skin, comprising modulation of the level of expression or the activity of a dermal gene, where said gene is selected from the list constituted by the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV and ACTN1. Alternatively, the gene may be selected from list A of dermal genes linked to the TGF-beta-SMAD signalling pathway, preferably without SEMA5A, or indeed from list B of dermal genes, preferably without ITGB1, or indeed from the various sub-groups of this latter list, namely from LEPREL1, PLOD2, COL6A3 and CRTAP, or indeed from LAMC1, LAMB3 and LAMA3, or indeed from FRAS1, MATN2 and DST, or again from ITGA2, ITGAV and ITGB1, or between ITGA2 and ITGAV.

The term “non-pathological cutaneous pigmentary spots” encompasses benign spots, which it is desirable to eliminate for aesthetic reasons alone and not for therapeutic reasons. Pigmentation irregularities include pigmentary imperfections rendering the complexion non-uniform or the skin colour non-homogeneous.

The present inventors have demonstrated the important role of the genes of the invention in the dermis of pigmentary spots, and in particular the link between deregulation of the level of expression of these genes and the appearance of pigmentary spots. For this reason, suspending or reducing the modulation of these genes means that the deregulations observed can be reduced or abolished, and thus a situation can be restored in the extracellular matrix which is compatible with the absence of pigmentary spots and thus with a uniform complexion and a homogeneous skin colour.

For the reasons given for the other aspects of the invention, preferably, more than one gene is selected from the genes of the invention, for example at least two genes, or at least three, five, six, or eight. In one embodiment, the cosmetic method is intended to modulate the level of expression of all of the genes of the invention, or all of the genes from list A, with or without SEMA5A, or indeed all of the genes from list B. Preferably, the gene ITGB1 is then excluded from list B. The cosmetic method of the invention is intended to restore the levels of expression to close to those observed in healthy skin, for example adjacent to a pigmentary spot. Particularly preferred genes are the genes TGFBR2, TGFBI, BMP2, SMAD3, THBS2 and TGFBR3, as well as the genes FRAS1, LEPREL1, MATN2, DST, PLOD2 and ITGA2.

In one preferred embodiment, said pigmentary spot is a hyperpigmentary spot, for example an actinic, senile or solar lentigo. In such a case, the desired modulation in the cosmetic methods of the invention is:
total, partial or temporary inhibition of the expression of at least one gene selected from the genes TGFBR2, TGFBI, BMP2, SMAD3, THBS2, TGFBR3, SEMA5A and SMAD7, or indeed from the genes FRAS1, LEPREL1, MATN2, DST, ITGA2, COL6A3, CRTAP, LAMC1, LAMB3, LAMA3, ITGAV, ITGB1 and ACTN1, or indeed from the genes TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, or indeed
an increase, possibly temporary, in the expression of the gene SOSTDC1, or of the gene PLOD2.

Preferably, the inhibition is not total inhibition but partial inhibition, tending to reduce the level of expression of the selected gene without in any way completely inhibiting its expression.

In accordance with another embodiment, said pigmentary spot is a hypopigmentary spot. In such a case, the desired modulation is the reverse of the previous situation; in particular, such a method aims to increase the expression of at least one gene selected from the genes TGFBR2, TGFBI, BMP2, SMAD3, THBS2, TGFBR3, SEMA5A and SMAD7, or indeed from the genes FRAS1, LEPREL1, MATN2, DST, ITGA2, COL6A3, CRTAP, LAMC1, LAMB3, LAMA3, ITGAV, ITGB1 and ACTN1, or indeed from the genes TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, or indeed to inhibit or reduce the level of expression of the gene SOSTDC1 or of the gene PLOD2.

The term “increase or reduction in the level of expression” includes increasing or reducing the degree of transcription of said genes, and increasing or reducing the degree of translation of said genes, as well as increasing or reducing the activity of proteins encoded by those genes.

Preferably, at the same time as modulating the level of expression which is desired for one of the genes of the invention, a cosmetic method of the invention preferably also comprises modulating at least one other dermal gene selected from the genes EFEMP1, ECM1, ASPN, HS3ST6, PAPLN, CHSY1, FLRT2, MXRA5, LYZ, CTSL2 and PLAU and TIMP1. Preferred genes from this second list of dermal genes are the genes EFEMP1, ECM1, ASPN, HS3ST6, PAPLN and CHSY1 and the genes MXRA5, LYZ, CTSL2, PLAU and TIMP1.

The modulations applied in the case of treatment of a hyperpigmentary spot are the reduction in the level of expression for the genes EFEMP1, ASPN, PAPLN, CHSY1, MXRA5, LYZ, PLAU and TIMP1, and the increase in the level of expression for the genes ECM1, HS3ST6, FLRT2 and CTSL2. In the case of treatment of a hypopigmentary spot, the converse modulations are applied.

A cosmetic method in accordance with the present invention thus comprises applying a product, in particular a chemical molecule, natural extract, nucleic acids, peptides or a treatment modulating the level of expression or activity of an expression product of at least one of the dermal genes of the invention.

The modulations are preferably obtained using an antisense, a microRNA or a siRNA directed against at least one of the genes of the invention and inhibiting its expression.

If it is a product, it is preferably applied topically.

Preferably, the modulation is carried out using a modulator of one of the genes of the invention. Such modulators are described in Example 3 and Table 3. As an example, a cosmetic method of the invention will advantageously comprise applying a compound selected from demineralized bone powder (DBP), Pioglitazone, GW0742, Cristata L flavonoid, Fenofibrate, Oxymatrine, salvianolic acid B, SB-431542, a Wen-pi-tang-Hab-Wu-ling-san extract, Tetrandrine and N-acetyl-seryl-aspartyl-lysyl-proline (Ac-SDKP), vitamin K2 menaquinone, beta-aminopropionitrile (bAPN) and Vandetanib (ZD6474 5), or indeed the application of low intensity ultrasound, or from associations of at least two of these modulators.

Furthermore, a cosmetic method of the invention is advantageously carried out after characterization of the pigmentary spot which is to be treated using a method in accordance with the first aspect. In fact, this first step can be used to characterize the spot and thus to detect the genes for which the level of expression is strongly modulated between the zone of the spot and an undamaged zone, preferably adjacent. It is then possible to adapt a treatment which can be used to act on the gene or genes of the invention which are differentially modulated in that spot, by specifically applying modulators for said genes.

Irrespective of the treatment considered, the cosmetic method of the invention may also comprise applying one or more additional active compounds intended to reinforce the desired effects, for example any substance described as being depigmenting, keratolytic and/or desquamating agents, antioxidants, chemical or physical UV sunscreens, anti-inflammatories and/or soothing agents, or deoxyribonucleic acids and their derivatives.

The cosmetic method of the present invention is also applicable in the case of preventing the appearance of pigmentary spots or other irregularities in the complexion or the skin colour, in particular hyperpigmentary spots such as actinic lentigo.

In fact, the data obtained by the inventors and set out in the experimental section reveal that challenges to the extracellular matrix and in particular to the TGF-β-SMAD signalling pathway by means of the genes brought to light by the inventors are susceptible of occurring before the spots appear. The sequence of biological events in the hyperpigmentary spots could be considered to be as follows: 1) alteration to the dermis (due to modulation of the expression of the genes identified in this application), which results in 2) modification to the epidermis with formation of epidermal ridges in the dermis, which results 3) in an increase in the length of the dermoepidermal junction and the formation of complex networks, resulting in 4) an increase in melanic load.

The present invention also concerns the use of a modulator of the level of expression or activity of the expression product of at least one dermal gene selected from the list constituted by the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV, ITGB1 and ACTN1, for a cosmetic application in the treatment of non-pathological cutaneous pigmentary spots or, more generally, in the modulation of skin pigmentation. Alternatively, the gene may be selected from list A of dermal genes linked to the TGF-beta-SMAD signalling pathway, with or without SEMA5A, or indeed from list B of dermal genes, or indeed from the different sub-groups of this latter list, namely from LEPREL1, PLOD2, COL6A3 and CRTAP, or indeed from LAMC1, LAMB3 and LAMA3, or indeed from FRAS1, MATN2 and DST, or again from ITGA2, ITGAV and ITGB1, or between ITGA2 and ITGAV. ITGB1 can be excluded from the various lists.

For the treatment of hyperpigmentary spots, the modulator used is an inhibitor of at least one gene selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1 or indeed an activator of the gene SOSTDC1 or of the gene PLOD2. Preferably, the inhibitor is a partial inhibitor leading to a reduction in the level of expression or to a reduction in the activity of the expression product of said gene, without in any way completely stopping the expression or activity of that gene. In one aspect, the modulator is an inhibitor of at least one gene selected from TGFBR2, TGFBI, BMP2, SMAD3, THBS2, TGFBR3, SEMA5A and SMAD7, or without SEMA5A, or indeed an activator of SOSTDC1. In another aspect, the modulator is an inhibitor of at least one gene selected from FRAS1, LEPREL1, MATN2, DST, ITGA2, COL6A3, CRTAP, LAMC1, LAMB3, LAMA3, ITGAV and ACTN1, or indeed an activator of PLOD2.

In contrast, for the treatment of hypopigmentary spots, the modulator used is an activator of at least one gene selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, or indeed an inhibitor of the gene SOSTDC1 or of the gene PLOD2. In one embodiment, the modulator is an activator of at least one gene selected from TGFBR2, TGFBI, BMP2, SMAD3, THBS2, TGFBR3, SEMA5A and SMAD7, or without SEMA5A, or indeed an inhibitor of SOSTDC1. In another aspect, the modulator is an activator of at least one gene selected from FRAS1, LEPREL1, MATN2, DST, ITGA2, COL6A3, CRTAP, LAMC1, LAMB3, LAMA3, ITGAV and ACTN1, or indeed an inhibitor of PLOD2.

Examples of known and characterized inhibitors or activators are disclosed in Example 3 of the experimental section. Such modulators may in particular be salvianolic acid B, Wen-pi-tang-Hab-Wu-ling-san extract or low intensity ultrasound pulses, for use in the cosmetic treatment of hyperpigmentary spots.

Well known modulators are also antisense molecules, siRNAs, and microRNAs. Particular envisaged modulators are antisense molecules, microRNAs and siRNAs directed against at least one of the genes of the invention and inhibiting its expression.

Particular preferred modulators in the context of the present invention are demineralized bone powder (DBP), Pioglitazone, GW0742, Cristata L flavonoid, Fenofibrate, Oxymatrine, salvianolic acid B, SB-431542, a Wen-pi-tang-Hab-Wu-ling-san extract, Tetrandrine, N-acetyl-seryl-aspartyl-lysyl-proline (Ac-SDKP), vitamin K2 menaquinone, beta-aminopropionitrile (bAPN) and Vandetanib (ZD6474 5), or indeed the application of low intensity ultrasound. The use of a combination of at least two of these modulators may also be envisaged.

The effective quantities of the modulators should be adapted as a function of the desired result and the type and number of modulators used; they may be in the range 0.001% to 30% by weight.

Modulators such as those described may be used in the cosmetic methods of the invention in association with modulators of the genes EFEMP1, ECM1, ASPN, HS3ST6, PAPLN, CHSY1 and FLRT2, or indeed modulators of the genes MXRA5, LYZ, CTSL2, PLAU and TIMP1.

Preferred combinations of the genes have already been described.

The modulators may be used in association with other products, active ingredients or excipients. Preferably, they are packaged in a suitable form for topical application, for example in the form of an ointment, cream or salve, or in any form suitable for skincare, such as a lotion, serum, soap, etc.

The various implementations detailed in the section relating to the cosmetic methods of the invention are applicable to uses for the cosmetic applications described above.

The modulators of the invention may in particular be used in cosmetic applications with a view to evening out the complexion, homogenizing the skin colour or combating dyschromias.

Furthermore, in another aspect the present invention concerns modulators of the level of expression or the activity of an expression product of at least one dermal gene selected from the genes TGFBR2, TGFBI, BMP2, SMAD3, THBS2, TGFBR3, SEMA5A, SMAD7, SOSTDC1, FRAS1, LEPREL1, MATN2, DST, PLOD2, ITGA2, COL6A3, CRTAP, LAMC1, LAMB3, LAMA3, ITGAV and ACTN1 for application in the treatment of cutaneous pigmentary spots, for example in the context of therapeutic treatments. Said spots may be hyperpigmentary spots, or indeed hypopigmentary spots. Alternatively, the gene may be selected from list A of dermal genes linked to the TGF-beta-SMAD signalling pathway, or indeed from list B of dermal genes, or indeed from the different sub-groups of this latter list, namely from LEPREL1, PLOD2, COL6A3 and CRTAP, or indeed from LAMC1, LAMB3 and LAMA3, or indeed from FRAS1, MATN2 and DST, or again from ITGA2, ITGAV and ITGB1, or between ITGA2 and ITGAV.

Preferred modulators have already been described in respect of other aspects of the invention.

They may be associated with other products, active ingredients or excipients. Preferably, they are packaged in a suitable form for topical application, for example in the form of an ointment, cream or salve, or in any form suitable for skincare such as a lotion, serum, soap, etc.

In the various methods and applications in accordance with the present invention, the pigmentary spots under consideration are preferably hyperpigmentary spots and more preferably actinic, solar or senile lentigines. The methods and applications of the invention are based on the demonstration by the inventors of a signature for such pigmentary spots.

This signature is characterized by the fact that it can be constituted by the whole list or a portion of the cited genes.

The present invention also pertains to the use of this signature as a novel method for selecting and predicting actinic lentigo through deficiencies in its biological functions. This novel method is based on a study of the level of expression of all or a part of the genes described in the present invention in a hyperpigmented lesion as opposed to an adjacent undamaged skin.

This invention also falls within the context of the treatment of actinic lentigo by using these genes as a molecular signature of differences in gene expression existing between a lesion and adjacent healthy skin. This signature also constitutes a clear advantage in determining the choice of appropriate treatment and measuring the effect of a product (active ingredient, molecule, natural extract), but also of a method (light, injection, orally) which is supposed to be beneficial to the skin. The present invention can in fact be used to evaluate the efficacy of a product or method intended to treat actinic lentigo by modulating the level of expression in the lesion of all or a portion of the genes described such that their expression profile is close to that of healthy skin.

The invention also consists in a method for screening inhibition or prevention factors for actinic lentigines. It consists of evaluating compounds for their power to inhibit or increase expression of the cited genes and/or the expression or activity of the protein products from said genes and to select those factors which can prevent or treat the actinic lentigo. Verification of the efficacy of the compounds may be carried out on mono or co-culture models or three-dimensional models of reconstructed skin, or on ex vivo skin or on skin in vivo.

The invention also pertains to the use of compounds modulating the expression of genes identified from biomarkers of actinic lentigo in order to prevent or correct the lesion in order to restore the skin to its normal state, i.e. to restore expression approaching the expression of a healthy undamaged skin. In particular, compounds acting on proteins identified for preventing or treating pigmentary dyschromia (hyperpigmentation or hypopigmentation) have never before been proposed. Such compounds exist and are reported in Table 3 of Example 3.

The selected agents are in particular negative modulators of proteins overexpressed with respect to the extracellular matrix, in particular proteins of the TGF-β-SMAD signalling pathway, or positive modulators of underexpressed proteins.

Particular negative modulators of proteins linked to the extracellular matrix and in particular to the TGF-β-SMAD signalling pathway which can be cited include inhibitors of synthesis and/or secretion and/or activators of the degradation of proteins which are found to be overexpressed in lentigo.

In contrast, positive modulators which can be cited are synthesis stimulants, secretion inducers or inhibitors of the degradation of proteins underexpressed in lentigo.

### KEY TO FIGURES

FIG. 1: This figure illustrates the Reconstructed Pigmented Skin (RPS) treatment protocol using TGF-β. In the diagram, T means “treatment with TGF-β”, in a concentration of 200 pg/mL, with 4 mM of HCl and 1 mg/mL of BSA as the vehicle. The medium was MEM 3F′ with 2% FBS. The equivalent derma were treated for 3 consecutive days. On D0i, the equivalent dermis was seeded with keratinocytes and melanocytes (K+M on the graph) and kept immersed for 7 days. The culture was then emersed for 2 weeks;

FIG. 2: This figure represents photographs of reconstructed pigmented skin after treatment with a control (2% SVF HCl/BSA) or with TGFbeta (200 pg/mL) using the protocol described in Example 4. The two upper photographs correspond to the Dopa reaction on detached epidermis. The two lower photographs correspond to Fontana Masson staining;

FIG. 3: This figure illustrates the luminance results (graphs A and C) and melanin quantification (graphs B and D), for reconstructed pigmented skin treated in accordance with the protocol described in Example 4. The control corresponds to a treatment with 2% SVF HCl/BSA, TGFB corresponds to a treatment with TGF-beta in a concentration of 200 pg/mL. The upper 2 graphs and the lower 2 graphs correspond to two identical and independent experiments.

### EXPERIMENTAL SECTION

### Example 1

### Transcriptional Study

A comparative study of the gene expression profile of skin obtained from an actinic lentigo (LS) lesion and adjacent undamaged skin (US) was carried out.

The aim of this study was to identify pertinent, reproducible and significant markers reflecting the changes associated with the formation of actinic lentigo in order to use them as targets for effective treatments or as biomarkers to analyse the efficacy of a given treatment.

In brief, 15 female volunteers were recruited to participate in a “full genome” transcriptome study (Affymetrix arrays). For each volunteer, an actinic lentigo type lesion was diagnosed on the back of the hand and the actinic lentigo diagnosis was confirmed by epiluminescence. This examination was in order to:

1°) verify the clinical diagnosis of a lesion (exclusively actinic lentigo) using the dermoepidermal junction pattern criteria (“fingerprint-like structure”) to be differentiated from ephelides (absence of fingerprint-like structure, homogeneous pigmentation and moth-eaten edge zones) and from flat sebborrheic keratoses (multiple milia-like cysts or pseudocysts and moth-eaten edge zones, pseudofollicular openings and fingerprint-like pattern) [Menzies et al; Stolz et al; Carli et al];
2°) define homogeneous zones in structure/pattern terms inside these lesions where skin biopsies were to be carried out;
3°) establish a phenotype score based on a quantitative image analysis using specific software developed on Matlab® (SQA software, CMLA, ENS Cachan, UMR CNRS 8536).

A 3 mm biopsy centred on the lesion was taken as well as an identical size biopsy on an adjacent undamaged skin zone. The total RNA from these samples was extracted and amplified. Probes were generated for hybridization on Affymetrix arrays. The gene expression profiles were generated for each of the 30 biopsies (2 biopsies per volunteer) and a comparative analysis was carried out between US and LS, per volunteer and over all of the volunteers. The genes which were statistically differentially expressed (geometric mean of patients) were compiled into lists and grouped into functional families.

Surprisingly and unexpectedly, the inventors found a differential expression for several hundred genes between US and LS. A list of 437 genes was drawn up and represents a broad molecular signature of the actinic lentigo lesion. Of the 437 identified genes, 169 genes were shown to be positively regulated (upregulated) in the actinic lentigo lesion compared with healthy skin, while 269 genes were regulated in a negative manner in the actinic lentigo (downregulated).

This group of genes was subdivided into a plurality of functional families.

Of the functional families or biological processes identified, the inventors surprisingly discovered a family of genes reflecting, on a molecular level, a dysfunction in the extracellular matrix and the dermoepidermal junction in the actinic lentigo. These genes, collected into this functional family known as the “extracellular matrix”, have never before been described as being associated with actinic lentigo and are listed below and in Tables 1 and 2:

Genes overexpressed in actinic lentigo compared with healthy skin TGFBR2, TGFBI, BMP2, SMAD3, THBS2, TGFBR3, SEMA5A, SMAD7, EFEMP1, ASPN, PAPLN, CHSY1, MXRA5, LYZ, PLAU, TIMP1, FRAS1, LEPREL1, MATN2, DST, ITGA2, COL6A3, CRTAP, LAMC1, LAMB3, LAMA3, ITGAV, ITGB1 and ACTN1.
Genes underexpressed in actinic lentigo compared with healthy skin SOSTDC1, ECM1, HS3ST6, FLRT2, CTSL2 and PLOD2.

These genes code for components of the dermis or for proteins involved in the synthesis of components of the extracellular matrix linked to renewal or remodelling this connective matrix, as well as to genes coding for matrix proteins associated with the dermoepidermal junction and the basal membrane zone. This family also contains genes linked to the TGF-β pathway, involved in the synthesis of components of the extracellular matrix. The genes of this family are mainly overexpressed in actinic lentigo. This family of genes is entirely original in a pigmentary disorder and points to a preponderant role for stroma in actinic lentigo.

TABLE 1
List of genes of the “extracellular matrix” family found to be overexpressed in
actinic lentigo

| Sub-family | | | | |
|---|---|---|---|---|
| pathway/ | | Accession | | Degree of |
| function | Denomination | number | Full name | modulation |
| TGFb/ | THBS2 | NM_003247 | thrombospondin 2 | 3.41 |
| SMAD | TGFBI | NM_000358 | transforming growth factor, | 2.22 |
| | | | beta-induced, 68 kDa | |
| | BMP2 | NM_001200 | bone morphogenetic protein 2 | 2.03 |
| | SMAD3 | NM_005902 | SMAD family member 3 | 1.76 |
| | TGFBR3 | NM_003243 | Transforming growth factor, | 1.68 |
| | | | beta receptor III | |
| | TGFBR2 | D50683 | transforming growth factor, | 1.61 |
| | | NM_001024847 | beta receptor II (70/80 kDa) | |
| | | NM_003242 | | |
| | SEMA5A | NM_003966 | sema domain, seven | 1.56 |
| | | | thrombospondin repeats | |
| | | | (semaphorin) | |
| | | | (semaphorin 5A, semaphorin | |
| | | | F) | |
| | SMAD7 | NM_005904 | SMAD family member 7 | 1.52 |
| Collagens | LEPREL 1 | NM_018192 | leprecan-like 1 | 2.64 |
| | COL6A3 | NM_004369 | collagen, type VI, alpha 3 | 1.90 |
| | CRTAP | NM_006371 | cartilage associated protein | 1.60 |
| | | | (LEPREL3) | |
| Laminins | LAMC1 | NM_002293 | laminin, gamma 1 (formerly | 1.96 |
| | | | LAMB2) | |
| Laminins | LAMB3 | L25541 | laminin, beta 3 | 1.94 |
| | | NM_000228 | | |
| | | NM_001017402 | | |
| | | NM_001127641 | | |
| | LAMA3 | NM_000227 | laminin, alpha 3 | 1.64 |
| Integrins | ITGA2 | NM_002203 | integrin, alpha 2 (CD49B, | 1.62 |
| | | | alpha 2 subunit of VLA-2 | |
| | | | receptor) | |
| | ITGAV | NM_001145000 | integrin, alpha V (vitronectin | 1.62 |
| | | NM_001144999 | receptor,) | |
| | | NM_002210 | | |
| | ITGB1 | NM_133376 | integrin, beta 1 | 1.55 |
| Matrix | MXRA5 | NM_015419 | matrix-remodelling associated 5 | 2.40 |
| remodelling | LYZ | NM_000239 | lysozyme (renal amyloidosis) | 2.33 |
| | TIMP1 | NM_003254 | TIMP metallopeptidase | 2.26 |
| | | | inhibitor 1 | |
| | PLAU | NM_002658 | plasminogen activator, | 1.81 |
| | | | urokinase | |
| Proteoglycans | EFEMP1 | NM_001039348 | EGF-containing fibulin-like | 3.86 |
| and | | NM_001039349 | extracellular matrix protein 1 | |
| extracellular | | NM_004105 | (FIBULIN 3) | |
| glycoprotein | ASPN | NM_017680 | Asporin | 2.77 |
| | PAPLN | NM_173462 | papilin, proteoglycan-like | 2.51 |
| | | | sulfated glycoprotein | |
| | CHSY1 | NM_014918 | carbohydrate (chondroitin) | 1.50 |
| | | | synthase 1 | |
| Basal | FRAS1 | NM_025074 | Fraser syndrome 1 | 4.59 |
| membrane | MATN2 | NM_002380 | matrilin 2 | 2.61 |
| | DST | NM_001723 | Dystonin = BPAG1 | 2.24 |
| | | NM_015548 | | |
| Actin | ACTN1 | NM_001130005 | actinin, alpha 1 | 1.58 |
| | | NM_001130004 | | |
| | | NM_001102 | | |

TABLE 2
List of genes of the “extracellular matrix” family found to be underexpressed in
actinic lentigo

| Sub-family | | | | |
|---|---|---|---|---|
| pathway/ | | Accession | | Degree of |
| function | Denomination | number | Full name | modulation |
| Proteoglycans | HS3ST6 | NM_001009606 | heparan sulfate (glucosamine) | 0.44 |
| | | | 3-O-sulfotransferase 6 | |
| Collagens | PLOD2 | NM_000935 | procollagen-lysine, 2- | 0.45 |
| | | NM_182943 | oxoglutarate 5-dioxygenase 2 | |
| Proteoglycans | FLRT2 | NM_013231 | fibronectin leucine rich | 0.47 |
| | | | transmembrane protein 2 | |
| Matrix | CTSL2 | NM_001333 | cathepsin L2 (peptidase MEC | 0.54 |
| remodelling | | | Degradation) | |
| Proteoglycans | ECM1 | U65932 | extracellular matrix protein 1 | 0.54 |
| | | NM_004425 | | |
| | | NM_022664 | | |
| TGFb/ | SOSTDC1 | NM_015464 | sclerostin domain containing 1 | 0.62 |
| SMAD | | | (ectodin, BMP antagonist) | |

### Example 2

### Material and Methods

15 female volunteers with phototypes II to IV aged 50 to 70 years were selected. Actinic lentigines from the back of the hand with a minimum dimension of 3 mm were selected. They were characterized by epiluminescence. The various advantages of the characterization by epiluminescence were presented in Example 1.

Two 3 mm diameter biopsies were taken from one of the hands of each patient. For each volunteer, one of the biopsies corresponded to the actinic lentigo lesion (LS) and the other to an adjacent undamaged zone of the skin (US) (also verified by epiluminescence).

The 3 mm biopsies were placed, from the time of sampling, in RNAlater (Qiagen reference 76106) for 16 to 24 h at 4° C. The next day, the samples were placed at −20° C. awaiting the homogenization and extraction steps. Upon defrosting, the samples were cut with a scalpel to facilitate homogenization before transfer into lysis buffer.

Homogenization was carried out with a Potter homogenizer (Fisher Labosi ref A6391000) with RNase free polypropylene plungers (Fisher Labosi ref A1419753) to allow direct homogenization in 1.5 mL Eppendorf tubes.

The RNA was extracted with RNeasy micro kits (Qiagen ref: 74004), following the manufacturer's instructions. RNA quantification was carried out by ribogreen assay (Molecular Probes ref R11490). The quality was confirmed with an Agilent 2100 bioanalyser, which provided a ratio of the intensities of 28S to 18S ribosomal RNA as well as the RNA Integrity Number (RIN), which takes RNA degradation into account. Good quality RNA has a ratio >1.5 and a RIN of >7.

A reverse transcriptase (RT) reaction was carried out to obtain the corresponding cDNA. Two probes per sample were synthesized from 50 ng of RNA with an amplification step. The cDNA was labelled with fluorochromes and hybridized onto Affymetrix® DNA chips in order to reveal the level of expression of all of the genes of the human genome. (Affymetrix U133A 2.0 U133A 2.0 type DNA bioarrays containing 54000 probes, allowing the expression of 47000 transcripts to be studied, including 38500 characterized genes). The Affymetrix Microarray suite (Mas 5.0) was used to obtain a detection signal for each transcript. After revealing specific hybridizations and processing the raw data (extraction, subtraction of background noise, normalization), gene expression was compared between healthy skin and damaged skin.

2 Affymetrix HG_U133 Plus 2 arrays were hybridized per sample.

The quality of the hybridization was ascertained using the AffyPLM method (Bolstad et al., 2005) and using the PCA (principal component analysis) method.

The patients were only retained for the remainder of the analysis if the 2 Affymetrix arrays had the correct hybridization quality. For the present study, 13 patients out of the initial 15 were able to be analysed.

Carrying out a transcriptome profile of healthy skin and skin corresponding to actinic lentigo meant that lists of genes expressed differentially in the two situations could be generated and biomarkers for actinic lentigo could be identified. The lists were generated in the form of the expression ratio between LS (lesional skin) versus US (undamaged skin). The ratio representing the geometric mean of 13 patients was retained.

Generation of lists of genes expressed differentially between damaged skin and healthy skin.

### Steps:

Filter for Affymetrix identifiers (probe sets): only the probe sets which are present in the two replicates of at least one biopsy were retained. After this filter, 23 968 probe sets were retained.
Suppressing patient effect: In order to suppress the patient effect observed in the results of the differential analyses, the expression of each probe set was divided by the geometric mean of the 4 values for the probe sets corresponding to the 4 arrays.
Differential analysis: This was generated by combining the lists obtained by 2 methods, the cNMF analysis (consensus Non Negative Matrix Factorisation) (Lee and Seung (1999), Brunet et al. (2004), Fogel et al. (2007), Fogel et al. (2008)), which had identified 2638 probe sets (including 1521 induced and 1117 repressed) and the PLS (Partial Least Squares Regression) method, which had identified 610 modulated probe sets. Combining the 2 lists produced a list of 3248 probe sets.
Filter for modulation: for the induced genes, selection of probe sets with a geometric mean of 13 corrected folds (CF)≧1.5: list of 562 induced probe sets. For the repressed genes, selection of probe sets with a geometric mean of 13 corrected folds CF≦0.67: list of 807 repressed probe sets. In total: 562+807=1369 modulated probe sets, i.e. 1007 probe sets after eliminating duplications (1002 cNMF approach+5 PLS approach)
Filter for 13 patients: visualisation of modulations of 1007 probe sets in the form of histograms and selection of probe sets modulated in the same sense in the 13 patients. Final list of 132 probe sets which differentiate the lesional biopsies from the undamaged biopsies and which are modulated in the same sense in the 13 patients of the study.
Analysis of list of 1007 probe sets
   Filter for P value (≦0.00001)
      In order to retain only the most discriminating genes, we applied to the list of 1002 Probe sets a filter for the P-value, P≦0.00001. This new filter produced 827 probe sets to which the 5 probe sets obtained from the PLS were added, [symbol] list of 832 probe sets.

After annotation search, elimination of non-annotated genes and elimination of duplications, a list of 437 genes expressed differentially between LS and US was established. 169 genes were overexpressed in AL and 269 were underexpressed.

Using the Gene Ontology, PubMed, and Scopus tools, the functions of the genes were investigated and the genes were classified into functional families.

### Example 3

### Modulators

Known compounds exist for modulating the proteins deregulated in lentigo in the desired sense. The following can be cited for the genes/proteins family of the extracellular matrix:
fibrates including fenofibrate, for example, which reduces the expression of SMAD3,
alkaloids including oxymatrine, which reduces SMAD3, or polyphenols such as, for example, salvianolic acid B which reduces SMAD3 and TGFBR2,
natural extracts, in particular medicinal herbs such as Wen-pi-tang-hab-wu-ling-san, which reduces SMAD3,
compounds of the imidazole family, which are antagonists of TGF-βR1, such as SB-431542 which reduces SMAD3,
certain miRNAs, in particular miR183 and miR29b, which reduce ITGB1,
inhibitors of urokinase-type plasminogen activator (uPA), such as p-aminobenzamidine or B428 4-substituted benzo[b]thiophene-2-carboxamodine which reduces the activity of PLAU,
compounds of the phenylacetates family, such as NaPA, which reduces the expression of PLAU,
compounds of the thiazolidinediones chemical family, including pioglitazone, which reduces BMP2 and COL6A3,
compounds of the thiazoles chemical family including, for example, GW-0742, which reduces the expression of BMP2,
compounds of the tetrazoles family, such as valsartan, which reduces TIMP1.

Concerning the enzymatic protein PLOD2 of the extracellular family which is underexpressed in lentigo, the following compounds may be employed to restore its levels:
compounds of the quinazolines family, such as vandetanib (ZD64745 5), which increases PLOD2.

Other types of modulators may also be employed in order to correct the expression/quantity or activity of deregulated proteins. The following may be cited:
nucleic acids (preferably antisense RNA or RNAi), neutralizing antibodies,
electrical, light, mechanical or thermal means. As an example, low intensity pulsed ultrasound (LIPUS) could be used to increase the quantity or activity of PLOD2.

In contrast, in the case of a hypopigmentary spot, preferred modulators will be those increasing the level of expression or activity of proteins obtained from the genes TGFBR2, TGFBI, BMP2, SMAD3, THBS2, TGFBR3, SEMA5A, SMAD7, EFEMP1, ASPN, PAPLN, CHSY1, MXRA5, LYZ, PLAU, TIMP1, FRAS1, LEPREL1, MATN2, DST, ITGA2, COL6A3, CRTAP, LAMC1, LAMB3, LAMA3, ITGAV, ITGB1 and ACTN1 and those reducing the level of expression or activity of proteins obtained from the genes SOSTDC1, ECM1, HS3ST6, FLRT2, CTSL2 and PLOD2.

Examples of such modulators are also listed in Table 3.

TABLE 3
LIST OF GENE MODULATORS INVOLVED IN ACTINIC LENTIGO

| Sub- | | | Sense of |
|---|---|---|---|
| family/function | Name | Gene/protein modulator | modulation |
| TGFB/ | TGFBI, transforming growth | Demineralized bone powder (DBP) | Increases |
| SMAD | factor β-induced, 68 kDa | | expression |
| TGFB/ | BMP2, bone morphogenetic | Pioglitazone | Reduces exp. |
| SMAD | protein 2 | GW0742 | Reduces exp. |
| | | Cristata L flavonoid | Increases exp. |
| TGFB/ | SMAD3, SMAD family member 3 | Fenofibrate | Reduces |
| SMAD | | Salvianolic Acid B (Sal B), component of Danshen (a traditional | expression |
| | | Chinese herb used for chronic kidney disease), antioxidant and | |
| | | cellular protection. | |
| | | SB-431542 (a specific inhibitor of TβR-I kinase) | |
| | | Wen-pi-tang-Hab-Wu-ling-san (WHW) extract | Reduces |
| | | | phosphorylation |
| TGFB/ | TGFBR2, transforming growth | Salvianolic acid B (SA-B) | Reduces |
| SMAD | factor, β receptor II (70/80 kDa) | | expression |
| TGFB/ | SMAD7, SMAD family member 7 | Oxymatrine | Increases |
| SMAD | | 3-deoxyglucosone (Advanced glycation end product precursor) | expression |
| | | Tetrandrine | |
| | | N-acetyl-seryl-aspartyl-lysyl-proline(Ac-SDKP) | |
| Collagen | COL6A3, collagen, type VI, | Pioglitazone | Reduces |
| | alpha 3 | | expression |
| Integrin | ITGB1, integrin, beta | miR-183, miR-29b | Reduces |
| | | | expression |
| Matrix | LYZ, lysozyme (renal | Pituitary adenylate cyclase-activating polypeptide (PACAP) | Increases |
| remodelling | amyloidosis) | PACAP38 | expression |
| Matrix | TIMP1, | Valsartan, (angiotensin II type 1 receptor blocker ARB) | Reduces |
| remodelling | metallopeptidase inhibitor 1 | Plant extract from Lupinus albus LU10 | expression |
| | | Demineralized bone powder (DBP) | Increases exp. |
| Matrix | PLAU, plasminogen activator, | Sodium phenylacetate (NaPA) | Reduces |
| remodelling | urokinase | | expression |
| | | P-aminobenzamidine (Urokinase-type plasminogen activator(uPA) | Reduces activity |
| | | inhibitor): B428 4, B392- | |
| | | substituted benzo[b]thiophene-2-carboxamidines (Urokinase-type | |
| | | plasminogen activator(uPA) inhibitor | |
| | | Thienopyridine SR 25989 (Angiogenesis inhibitor) esterified | Increases |
| | | derivative of ticlopidine, | expression |
| | | Notoginsenoside R1 (obtained from PANAX notoginseng) | |
| Assimilated | ASPN, Asporin | Letrozole, anastrozole | Increases |
| proteoglycan | | | expression |
| Basal | MATN2, matrilin 2 | Vitamin K2 menaquinone | Increases |
| membrane | | | expression |
| Collagen | PLOD2, | beta-aminopropionitrile (bAPN) | Reduces |
| | procollagen-lysine, 2- | | expression |
| | oxoglutarate 5-dioxygenase 2 | Low-intensity pulsed ultrasound (LIPUS) | Increases exp./ |
| | | Vandetanib (ZD6474 5) | activity |
| Matrix | CTSL2, cathepsin L2 | Phenylalanine derivatives | Reduces activity |
| remodelling | | | |

### Example 4

### Stimulation of Pigmentation of Reconstructed Pigmented Skin by Treatment with TGF-Beta

1) Treatment Protocol.

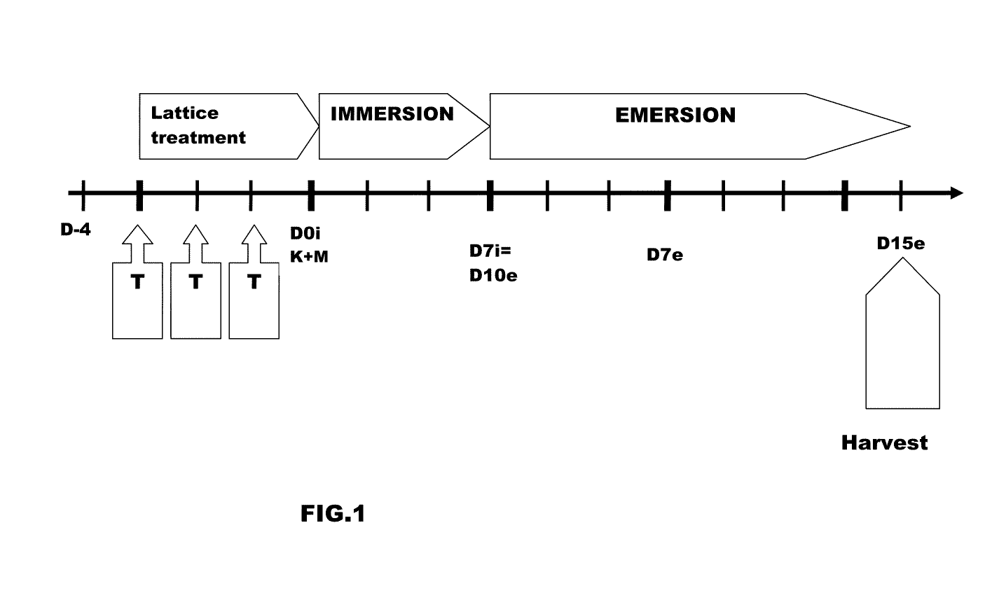

The treatment protocol for reconstructed pigmented skin (RPS) with TGF-β is illustrated in FIG. 1. It was as follows:

Equivalent dermis (lattices) containing live fibroblasts in a collagen matrix were attached to the bottom of a Petri dish (D-4) and treated with TGF-β1 (T on FIG. 1) in a concentration of 200 pg/mL (vehicle: 4 mM of HCl and 1 mg/mL of BSA; medium: MEM 3F′ with 2% FBS), for 3 days (D-3; D-2 and D-1 in FIG. 1). Next, the pigmented epidermis was reconstructed on the equivalent dermis by seeding keratinocytes and melanocytes (D0). The culture was kept immersed for 1 week and emersed for 2 weeks (FIG. 1).

Samples were removed to analyse the pigmentation:
integration and morphology of melanocytes were observed after Dopa reaction on detached epidermis;
colorimetric luminance measurement provided information regarding the degree of clarity of the samples (the lower the L*, the darker the samples);
the presence of melanin was observed after Fontana Masson staining skin sections; and
the quantity of melanin present in the epidermis was measured by image analysis.

2) Results:

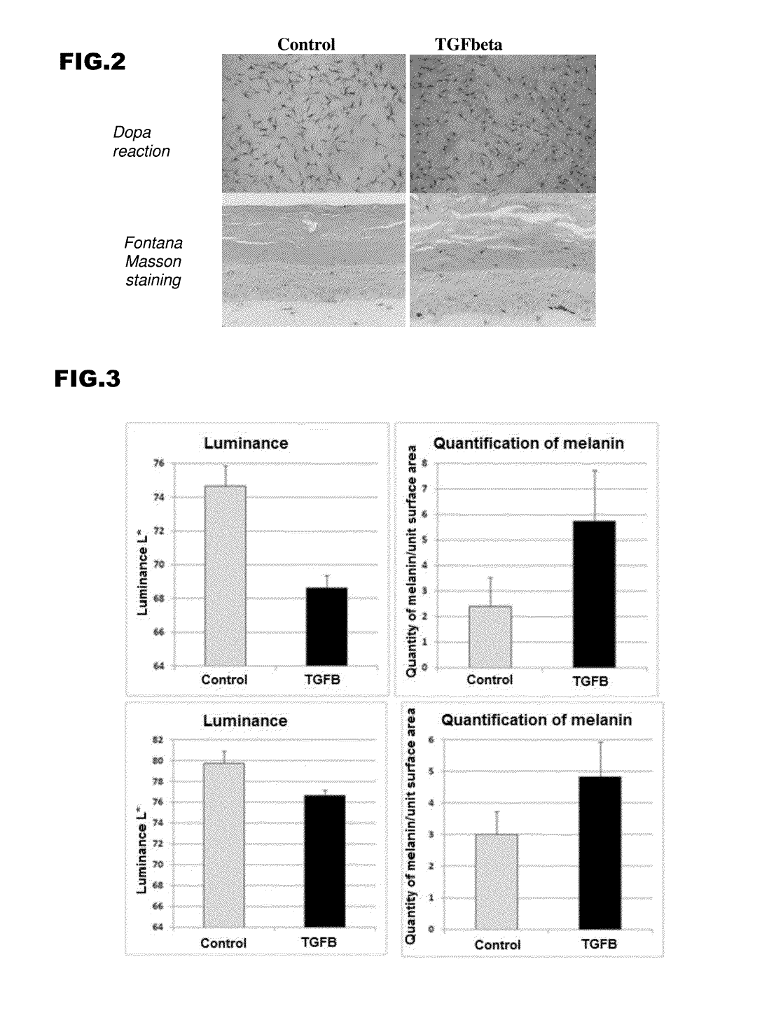

The results show that after treatment with 200 pg/mL of TGF-β1 and comparison with the control (FIGS. 2 and 3):
melanocytes are still present in the epidermis and retain the correct dendritic morphology;
melanin deposits are higher in the epidermis;
the luminance of the samples was reduced (browning of treated reconstructed pigmented skin);
the melanin content was visibly increased by observations of Fontana Masson stained sections and objectified by quantification of the melanin using image analysis.

The experiment was carried out a second time and confirmed the results obtained (FIG. 3).

The results demonstrate that activation of the TGF-β signalling pathway is responsible for greater pigmentation of the skin compared with an absence of activation of this signalling pathway.

### LIST OF REFERENCES

Andersen W K, Labadie R R, Bhawan J. “Histopathology of solar lentigines of the face: a quantitative study.” J Am Acad Dermatol. 1997 March; 36(3 Pt 1):444-7.
Ber Rahman S, Bhawan J. Lentigo. Int J Dermatol. 1996 April; 35(4):229-39. Review.
Berking C, Takemoto R, Schaider H, Showe L, Satyamoorthy K, Robbins P, Herlyn M. “Transforming Growth Factor-β1 Increases Survival of Human Melanoma through Stroma Remodelling”. Cancer Res (2001); 61: 8306-8316.
Cario-Andre M, Lepreux S, Pain C, Nizard C, Noblesse E, Taïeb A. “Perilesional vs. lesional skin changes in senile lentigo.” J Cutan Pathol. 2004 July; 31(6):441-7.
Carli P. Salvini C. “Lentigines including lentigo simplex, reticulated lentigo, and actinic lentigo.” In Color Atlas of melanocytic lesions of the skin. Soyer H. P., Argenziano G., Hofman-Wellenhof R., Johr R. Springer-Verlag Berlin Heidelberg 2007: 290-294.
Kawata Y, Suzuki H, Higaki Y, Denisenko O, Schullery D, Abrass C, Bomsztyk; “Bcn-1 Element-dependent activation of the laminin gamma 1 chain gene by the cooperative action of transcription factor E3 (TFE3) and SMAD proteins.” J Biol Chem. 2002; 277(13):11375-84.
Menzies S W, Crotty K A, Ingvar C, McCarthy W H. “Benign pigmented macules.” In An atlas of surface microscopy of pigmented skin lesions: Demoscopy. Eds Menzies S W, Crotty K A, Ingvar C, McCarthy W H. McGraw-Hill book company Australia Pty Limited, North Ryde, Australia. 2003: pp 53-60
Montagna W, Hu F, Carlisle K. “A reinvestigation of solar lentigines”. Arch Dermatol. 1980 October; 116(10):1151-4.
Stolz W, Braun-Falco O, Bilek P, Landthaler M, Burgdorf W H C, Cognetta A B. “Differential diagnosis of pigmented skin lesions” In Color atlas of dermatology. Eds Stolz W, Braun-Falco O, Bilek P, Landthaler M, Burgdorf W H C, Cognetta A B. Blackwell Wissenschafts-Verlag, Berlin, Germany. 2002: pp 41-66.
Verrecchia F, Chu M L, Mauviel A. “Identification of novel TGF-beta/SMAD gene targets in dermal fibroblasts using a combined cDNA microarray/promoter transactivation approach”. J. Biol. Chem. (2001) 276: 17058-17062.

1. A method for characterizing a known or suspected cutaneous pigmentary spot in a human being, comprising comparing levels of expression, in samples of skin obtained from said spot and from adjacent undamaged skin, of at least one dermal gene linked to the extracellular matrix selected from:
A. the list constituted by the genes TGFBR2, TGFBI, BMP2, SMAD3, THBS2, TGFBR3, SEMA5A, SMAD7 and SOSTDC1; or from
B. the list constituted by the genes FRAS1, LEPREL1, MATN2, DST, PLOD2, ITGA2, COL6A3, CRTAP, LAMC1, LAMB3, LAMA3, ITGAV, ITGB1 and ACTN1.

2. The method according to claim 1, comprising comparing the levels of expression of at least two distinct genes, preferably of at least five distinct genes selected from one and/or the other of lists A and B.

3. The method according to claim 1, wherein said gene is selected from the list constituted by the genes TGFBR2, TGFBI, BMP2, SMAD3, THBS2 and TGFBR3.

4. The method according to claim 1, wherein said spot is confirmed as a hyperpigmentary spot when the level of expression is:
higher in the skin sample obtained from the spot compared with the level in the sample of adjacent undamaged skin, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
lower in the skin sample obtained from the spot compared with the level in the sample of adjacent undamaged skin, if the gene is selected from SOSTDC1 and PLOD2.

5. The method according to claim 1, wherein said pigmentary spot is an actinic, senile or solar lentigo.

6. A method for evaluating the efficacy of a treatment for cutaneous pigmentary spots, comprising comparing the levels of expression in a skin sample obtained from said spot, before and after treatment, of at least one dermal gene linked to the extracellular matrix selected from the list constituted by the genes TGFBR2, TGFBI, BMP2, SMAD3, THBS2, TGFBR3, SEMA5A, SMAD7 and SOSTDC1, or indeed from the list constituted by the genes FRAS1, LEPREL1, MATN2, DST, PLOD2, ITGA2, COL6A3, CRTAP, LAMC1, LAMB3, LAMA3, ITGAV, ITGB1 and ACTN1.

7. The method according to claim 6, wherein said treatment is considered to be effective for the treatment of hyperpigmentary spots when the level of expression is:
lower after treatment compared with the level of expression before treatment, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
higher after treatment compared with the level of expression before treatment, if the gene is selected from SOSTDC1 and PLOD2; and
is considered to be effective for the treatment of hypopigmentary spots when the level of expression is:
higher after treatment compared with the level of expression before treatment, if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV, ITGB1 and ACTN1, and
lower after treatment compared with the level of expression before treatment, if the gene is selected from SOSTDC1 and PLOD2.

8. An in vitro method for evaluating the efficacy of a treatment of cutaneous pigmentary spots, comprising comparing, before and after treatment, the level of expression, in a cellular model representing the skin, of at least one dermal gene linked to the extracellular matrix selected from the list constituted by the genes TGFBR2, TGFBI, BMP2, SMAD3, THBS2, TGFBR3, SEMA5A, SMAD7 and SOSTDC1, or indeed from the list constituted by the genes FRAS1, LEPREL1, MATN2, DST, PLOD2, ITGA2, COL6A3, CRTAP, LAMC1, LAMB3, LAMA3, ITGAV, ITGB1 and ACTN1, or indeed the level of expression or activity of an expression product of said selected gene.

9. A cosmetic method for the treatment or prevention of a non-pathological cutaneous pigmentary spot of human skin, comprising modulating the level of expression or activity of a dermal gene linked to the extracellular matrix, where said gene is selected from the list constituted by the genes TGFBR2, TGFBI, BMP2, SMAD3, THBS2, TGFBR3, SEMA5A, SMAD7 and SOSTDC1 or indeed from the list constituted by the genes FRAS1, LEPREL1, MATN2, DST, PLOD2, ITGA2, COL6A3, CRTAP, LAMC1, LAMB3, LAMA3, ITGAV and ACTN1.

10. The method according to claim 9, wherein said method comprises the modulation of at least two genes, preferably of at least five distinct genes, selected from the list constituted by the genes TGFBR2, TGFBI, BMP2, SMAD3, THBS2, TGFBR3, SEMA5A, SMAD7 and SOSTDC1 and/or from the list constituted by the genes FRAS1, LEPREL1, MATN2, DST, PLOD2, ITGA2, COL6A3, CRTAP, LAMC1, LAMB3, LAMA3, ITGAV and ACTN1.

11. The method according to claim 9, wherein said pigmentary spot is a hyperpigmentary spot, preferably actinic, senile or solar lentigo, and in which said modulation is an inhibition if the gene is selected from TGFBR2, TGFBI, BMP2, SMAD3, THBS2, TGFBR3, SEMA5A, SMAD7, FRAS1, LEPREL1, MATN2, DST, ITGA2, COL6A3, CRTAP, LAMC1, LAMB3, LAMA3, ITGAV and ACTN1, and an increase in the level of expression or activity if the gene is selected from SOSTDC1 and PLOD2.

12. Use of a modulator of the level of expression or activity of an expression product of at least one dermal gene selected from the list constituted by the genes TGFBR2, TGFBI, BMP2, SMAD3, THBS2, TGFBR3, SEMA5A, SMAD7 and SOSTDC1, or from the list constituted by the genes FRAS1, LEPREL1, MATN2, DST, PLOD2, ITGA2, COL6A3, CRTAP, LAMC1, LAMB3, LAMA3, ITGAV and ACTN1, for a cosmetic application in the treatment of non-pathological cutaneous pigmentary spots, said modulator modifying the level of expression or activity of the expression product of the selected gene or genes.

13. Use according to claim 12, wherein said modulator is demineralized bone powder (DBP), Pioglitazone, GW0742, Cristata L flavonoid, Fenofibrate, Oxymatrine, salvianolic acid B, SB-431542, a Wen-pi-tang-Hab-Wu-ling-san extract, Tetrandrine or N-acetyl-seryl-aspartyl-lysyl-proline (Ac-SDKP), vitamin K2 menaquinone, beta-aminopropionitrile (bAPN) or Vandetanib (ZD6474 5), or the application of low intensity ultrasound or a combination of at least two of these modulators.

14. A modulator of the level of expression or activity of the expression product of at least one dermal gene selected from the list constituted by the genes TGFBR2, TGFBI, BMP2, SMAD3, THBS2, TGFBR3, SEMA5A, SMAD7 and SOSTDC1, or from the list constituted by the genes FRAS1, LEPREL1, MATN2, DST, PLOD2, ITGA2, COL6A3, CRTAP, LAMC1, LAMBS, LAMA3, ITGAV and ACTN1, for an application in the treatment of cutaneous pigmentary spots, said modulator modifying the level of expression or activity of the expression product of the selected gene or genes.

15. The modulator according to claim 14, selected from demineralized bone powder (DBP), Pioglitazone, GW0742, Cristata L flavonoid, Fenofibrate, Oxymatrine, salvianolic acid B, SB-431542, a Wen-pi-tang-Hab-Wu-ling-san extract, Tetrandrine, N-acetyl-seryl-aspartyl-lysyl-proline (Ac-SDKP), vitamin K2 menaquinone, beta-aminopropionitrile (bAPN) and Vandetanib (ZD6474 5), or the application of low intensity ultrasound.
